(19) United States
(12) Patent Application Publication
(10) Pub. No.: US 2019/0019573 A1
(43) Pub. Date: Jan. 17, 2019

(54) PATIENT CARE SYSTEM

(71) Applicant: ARES TRADING S.A., Aubonne (CH)

(72) Inventors: Colin LAKE, Boston, MA (US); Andrew PATERSON, Stirling Central Scotland (GB); Simon EXELL, Geneve (CH); Eric CHANIE, Geneve (CH); Georgios KOUVAS, Geneve (CH); John MULCAHY, Nyon (CH)

(21) Appl. No.: 15/555,710

(22) PCT Filed: Mar. 24, 2015
(86) PCT No.: PCT/IB2015/052164

§ 371 Date: Sep. 5, 2017

(51) Int. Cl. G16H 10/60; G16H 80/00; A61B 5/00

(52) U.S. Cl. CPC G16H 10/60; G16H 15/00; A61B 5/4842; G16H 80/00

(57) ABSTRACT

Patient care system comprising a medical device (1) for administering a medical treatment to a patient (15a) and a server system (6) configured to receive and transmit data via a communications network (16) to, respectively from users including patients (15a) and health care professionals (15b), the server system further configured to process and store data related to patient care. The server system comprises a database (6c) configured to encrypt and store encrypted data related to patient care, an application server (6b) including patient care software components for disease management (36) and patient information management (32), a communication server (6a) including a web server software application for data transfer through the internet, the patient care software components operable to receive medical device usage data comprising data on the usage of said medical device transferred through the communications network, and further operable to process said medical device usage data in conjunction with patient data (32c) to generate a report (32f) or a plurality of reports related to the treatment of the patient, the reports being accessible remotely via the communications network by registered users of the patient care system as a function of respective roles and privileges of the registered user stored in the server system.

### FIELD OF THE INVENTION

The present invention relates to an electronic system to monitor and assist in patient therapy and wellness, in particular patients suffering a chronic condition, such conditions including neurodegenerative diseases, particularly multiple sclerosis, endocrinological diseases, particularly growth hormone deficiency, and autoimmune diseases including rheumatoid arthritis, juvenile rheumatoid arthritis, psoriasis, plaque psoriasis, Crohn's disease, juvenile Crohn's disease, asthma, psoriatic arthritis, ulcerative colitis, systemic lupus erythematosus and ankylosing spondylitis. The present invention also relates to an electronic system to monitor and assist in patient therapy and wellness for patients suffering from cancer, in particular breast cancer.

Monitoring and assisting in patient therapy and wellness includes, in one of the specific aspects of the invention, monitoring the efficacy of a treatment regimen and in particular the efficacy of a dosage regimen.

### BACKGROUND

It is known to use an electronic injection device to administer a pre-determined dosing regimen. For example, WO 2005/077441, WO 2006/085175, WO 2006/085204 or WO 2007/088444 disclose an electronic injection device marketed under the registered trademarks RebiSmart and Easypod. Patient adherence to a pre-determined dosing regimen is often lower than 100%. This is because a patient may forget to inject or they may inject at the wrong time. Furthermore, a patient may intentionally omit injections due to pain or side effects of the medicament.

One limitation of known electronic injection devices is that it is not possible for the devices to measure the efficacy of the dosage regimen followed by the patient using the device. As such, the patient must recall from memory the extent to which they adhered to their pre-determined dosing regimen and report this information to a physician. The patient must also recall from memory the extent to which their dosing regimen affected their physiological state and report this information to a physician. The physician must then piece this information together to determine the extent to which the patient adhered to their pre-determined dosing regimen. The physician must also piece this information together to determine the extent to which adherence to the pre-determined dosing regimen influenced the patient's physiological state. It is possible that the patient may not be able to sufficiently recall or record their adherence to a pre-determined dosing regimen and the affect that their adherence had on their physiological state. This is particularly problematic in the case where a patient suffers from a neurodegenerative disordered, such as multiple sclerosis, where their memory may be affected. This problem is confounded by the fact that the patient's appointments with their physician may be spaced apart by prolonged periods of time, such as up to 6 months. Furthermore, the patient's appointments with the physician may be very short, e.g. less than 15 minutes long. As such, there may not be enough time during an appointment for a physician to accurately determine the efficacy of a treatment regimen. Thus, there is a need in the art for a system to determine the efficacy of a treatment regimen, such as a dosage regimen.

Patients suffering from chronic conditions, for instance neurodegenerative diseases or autoimmune diseases, usually meet their physician or other health care professionals (HCP) at regular intervals and may receive assistance and services from field nurses or other members of patient support organisations. As mentioned above the intervals may be spaced apart by prolonged periods of time that are not optimal to monitor the patient's condition, take corrective measures, provide optimal treatment, or provide accompanying therapeutic and wellness services as a function of the state of the patient's condition.

Many of the before mentioned diseases have a severe impact on the health-related quality of life, with somatic symptoms having a direct influence on emotional and social functioning. For instance, it is common for patients with chronic diseases to fall into a depression and experience relational problems. Additionally, the health-related quality of life may vary from individual to individual, depending on the level of somatic problems, personality traits, capability to mentally handle the disease and capability to maintain relationships and a social life. Compounding the issue of the negative impact of diseases such as Multiple Sclerosis on the health-related quality of life are the challenges faced by health care practitioners in managing patients due to high patient heterogeneity with regard to disease subtype, severity, comorbidities, symptoms, the impact of symptoms on the health-related quality of life, and the short-lived nature of symptoms. Often, standard clinical assessments do not provide enough information for HCPs to effectively manage the disease and improve health-related quality of life.

US2007/016443 discloses a medication compliance system and medical dosage device for treating chronic diseases such as hypertension, hypercholesterolemia and osteoporosis. The system and device in US2007/016443 aim to improve a patient's adherence to a dosage regime of a prescribed medication. The medical dosage device is provided with sensors to determine if and when a dose of medicament has been taken. A feedback system provides feedback information to the patient on the adherence to the prescribed dosage regime and a messaging system creates tailored messages to the patient through the most appropriate communication channel.

US2003/221687 discloses a system for treating depression which comprises different modules related to medicament treatment, cognitive measures and exercise therapy. However, neither US2007/016443 nor US2003/221687 are adapted to assess the correlation between a multitude of parameters defining a patient's physiological state and a medication regimen.

There is also a need for many diseases to gain a better understanding of the effects of the treatment regime on the health of the patient, this being difficult for chronic diseases, for instance Multiple Sclerosis and Growth Hormone deficiency, where the effects of drug administration have a large temporal offset from the time of administration. In these cases tracking the efficacy of a particular treatment regime is particularly difficult.

### SUMMARY OF THE INVENTION

An object of the invention is to provide a patient care system that improves patient treatment monitoring for health care professionals and that improves patient wellness and care.

It is advantageous to provide a patient care system that provides reliable, relevant and easily readable information for decision making for health care professionals (HCP).

It is advantageous to provide a patient care system that facilitates ease of administering medications for patients.

It is advantageous to provide a patient care system that provides easy health communication between HCPs and patients.

It is advantageous to provide a patient care system to provide accompanying therapeutic and wellness services as a function of the state of the patient's condition.

It is advantageous to provide a patient care system that enables reliable and economical tracking of the efficacy of a treatment regime, especially for chronic diseases, such as Multiple Sclerosis and Growth Hormone deficiency, where the effects of drug administration have a large temporal offset from the time of administration.

Objects of the invention have been achieved by the patient care system according to the independent claims. Various advantageous features of the invention are set forth in the dependent claims.

Disclosed herein is a patient care system comprising a medical device for administering a medical treatment to a patient and a server system configured to receive and transmit data via a communications network to, respectively from users including patients and health care professionals, the server system further configured to process and store data related to patient care. The server system comprises a database configured to encrypt and store encrypted data related to patient care, an application server including patient care software components for disease management and patient information management program, a communication server including a web server software application for data transfer through the internet, the patient care software components operable to receive medical device usage data comprising data on the usage of said medical device transferred through the communications network, and further operable to process said medical device usage data in conjunction with patient data to generate a report or a plurality of reports related to the treatment of the patient, the reports being accessible remotely via the communications network by registered users of the patient care system as a function of respective roles and privileges of the registered user stored in the server system. The patient care system advantageously further comprises a patient therapy component, including patient therapy software and/or hardware components having at least one interactive module selected from the group of physiological monitoring modules, psychological modules, training modules and information modules. The patient therapy component may further comprise patient therapy hardware components, such as wearable devices for monitoring stress, sensors that detect movement during sleep, and sensors and cameras for detecting motion during the physical training

The patient therapy software is preferably a web-based program comprising a module residing on the application server and a module residing on the patient's user interface device or on the medical device. Generated reports may advantageously comprise information generated from the patient therapy software.

Patient care software components may be configured to receive patient therapy data from the patient therapy module. The reports component may be further configured to display adherence to the compliance measurement to the patient therapy module.

In an embodiment, the patient care system may further comprise a central therapy software control module, configured to extract information generated from individual modules and identify needs for training modules.

In an embodiment, the patient therapy software and/or hardware components may comprise a cognitive remediation training module, the cognitive remediation training module being accessible from the user interface device (UID), the cognitive remediation training module comprising a processor configured to run a patient adapted program with training exercises, a results analyser and a memory to store past results.

In an embodiment, the patient therapy software and/or hardware components may comprise a cognitive depression management module, the cognitive depression management module being accessible from the user interface device (UID) and comprises a depression evaluation algorithm, a processor configured to run a patient adapted program with training exercises configured based on data from the depression evaluation algorithm, results analyser and a memory to store past results.

In an embodiment, the patient therapy software and/or hardware components may comprise a cognitive sleep management module, the cognitive sleep management module being accessible from the user interface device (UID) and comprises training exercises a results analyser and a memory to store past results.

In an embodiment, the processor is configured to iteratively adapt the cognitive training program during a training session, such that the program can change based on the information entered by the patient.

In an embodiment, the server system further comprises patient therapy software and/or hardware components including a physiological monitoring module, configured to measure the physiological state of a patient.

In an embodiment, the physiological monitoring module comprises at least one interactive physiological test selected from the group of vision tests, walking tests, mobility tests and muscular force test.

In an embodiment, the patient therapy software and/or hardware component includes a physiological parameter analysing module, configured to receive a measured health parameter from a biosensor, said physiological parameter analysing module being configured to read and interpret physiological data selected from body temperature, blood pressure, pulse rate, galvanic skin response, surface electromyography, electroencephalography measurements, oculography measurements, electrocardiography measurements, breathing sensor measurements, blood sugar sensor measurements, urine markers and blood markers.

In an embodiment, the biosensor is a wearable biosensor, preferably a bracelet or a patch biosensor which can be worn by the patient and is configured to continuously extract physiological data and transmit said data to the server system.

In an embodiment, the patient therapy software and/or hardware component comprises an activity tracking module configured to track an amount of exercise performed.

In an embodiment, the server system further comprises a rehabilitation training module and a processing unit to calculate the trajectory of markers located on a patient's body to assess gait for evaluating, planning and treating inability to walk in patients suffering from Multiple Sclerosis.

In an embodiment, the server system further comprises patient therapy software and/or hardware components including an information module configured to receive information requests from a user and to send responses on the user interface device (UID).

In an embodiment, the medical device comprises a reader configured to recognize the drug located inside from information on the drug container and a processing unit configured to send identification information about the drug to the remote server.

In an embodiment, the application server is configured to receive drug information from the user interface device or from health care professionals interface device and respond to the user interface device with drug related information.

In an embodiment, the information module comprises at least one downloadable instruction file and one video comprising instructions about use and administration of medication.

In an embodiment, the information module comprises point of interest data such as location and contact information of physicians, physiotherapists, support groups, the user interface device being configured receive an information request and to send a measured GPS location point to the information database in order to retrieve a point of interest.

In an embodiment, the information module is configured to display the location for the nearest toilet for patients with Crohn's syndrome.

In an embodiment, the information database comprises information about disease development and measurements, said information being displayed to the user interface device.

In an embodiment, the information module further comprises an online ordering system for organising the ordering and delivery of drugs or medical appliances. The online ordering system may be further configured to submit requests for health care insurance reimbursements and monitor reimbursements.

In an embodiment, the patient information management program further comprises calendar software components, and wherein said calendar software components are configured to prompt the patient to take drugs or take a patient therapy module.

In an embodiment, the calendar software components comprise a system of reminders and alerts, whereby reminders for next dose, appointments and training modules are sent to the display of the user interface device (UID).

In an embodiment, the patient information management program is configured to analyse entered data from patient outcome reports, training module and physiological measuring module and send triggers for further follow-up to a health care professional.

In an embodiment, the patient information management program comprises follow-up programs which can be selectively activated by a HCP, said follow-up programs comprising measurement of physiological parameters and pain ratings.

In an embodiment, the patient therapy software and/or hardware component comprises a diet module, including a database containing food information and being configured to receive a patient request of the suitability of a certain food for intake and respond to the patient with an indication of the suitability for intake.

In an embodiment, the patient care system comprises a quality of life monitoring module, measuring the patient's well-being against a scale.

In an embodiment, the patient information management program is configured to receive patient information submitted as free-text, such as information of additional drugs taken by the patient which are not comprised in the treatment regimen.

In an embodiment, the medical device is directly connected to the server system through the Internet.

According to a an aspect of the invention, the patient information management program comprises a reports component configured to generate reports, in the form of tables, charts, lists, diagrams or graphical representations based on information selected from drug administration history, adherence data, patient outcome reports, patient health reports, patient physiological data reports, medical device settings, treatment regimen data, and any combination of aforesaid information, and the reports component is configured to form composite reports for simultaneous display on a user interface device (UID) display, including composite adherence and patient outcome reports to facilitate evaluation of the effects of non-adherence to the treatment regimen or the efficacy of the medical treatment.

The present invention provides a holistic care system for assessing the efficacy of treatment and health-related quality of life, which enables health care professionals (HCPs) to improve medication and therapy in a patient's treatment regimen.

Another advantage of the present patient care system is that it not only enables HCPs, but also makes it possible for patients to self-monitor their treatments and the associated somatic and psychological effects, whereby the patients are more likely to become active in disease management and treatment decision-making, which in turn may lead to improved treatment compliance, and ultimately, improved outcomes.

A further advantage is that since the application does not limit patients with regards to the number of instruments completed or frequency of reporting, HCPs may configure the system to schedule certain PRO assessments at certain time intervals (e.g. monthly) for their interests. HCPs may therefore use the system to actively monitor patients.

The communication server may advantageously further comprise a remote service data upload software application configured for wireless telecommunication technology (WTT) data transfer using a cell phone network, to allow users with mobile devices such as smart phones or computer tablets comprising a wireless telecommunication technology (WTT) transmitter to connect remotely to the server system. The patient care system may advantageously further comprise a medical device connection station comprising a wireless telecommunication technology (WTT) transmitter configured for connection to the server system via a wireless telecommunications network, the medical device connection station configured to interconnect to the medical device and to upload medical device usage data to the patient information management program application via the WTT remote service data upload application on the server system. Alternatively, the medical device may incorporate a wireless telecommunication technology (WTT) transmitter configured for connection to the server system via a wireless telecommunications network, the medical device configured to directly upload medical device usage data to the patient information management program application via a WTT remote service data upload application on the server system.

In an embodiment, a client side software application may be installed on a mobile user interface device (UID) such as a phone or computer tablet, configured to upload medical device usage data to the patient information management program application via the WTT remote service data upload application on the server system.

The adherence to the treatment regimen may be represented by a first graphical representation, and the outcome report relating to a physiological state of a patient may be represented by a second graphical representation, the first and second graphical representations having a common time scale and displayed simultaneously on a graphical display of a user interface device such that an operator can compare the adherence to the treatment regimen and the physiological state of the patient.

The patient care system may advantageously further comprise a notification services software component configured to transmit email and/or SMS (Short Message Service) notifications to patients and optionally other users of the system. Notifications may include any one or more of alarms, visit reminders, treatment regime information, health information, and drug information.

The patient care system may advantageously further comprise patient therapy software and/or hardware components including tests, such as vision and walking tests, accessible or downloadable from the server system by users and especially patients, the software component configured to capture results of tests and feed them into the reports software component. Patient therapy software and/or hardware components may also include training exercises such as cognition training and mobility training. In an advantageous embodiment, patient therapy services may further be configured to receive data from sensors relating to physiological measurements of the patient's measured physiological data, for instance data captured automatically by sensors and transmitted to the server system via the patient's user interface device or medical device, or by the sensing or training device. Physiological data may for instance include any one or more of body temperature, blood pressure, pulse rate, weight, height, calories spent, galvanic skin response, surface electromyography, electroencephalography, oculography, breathing activity, muscle movement activity, blood sugar level, and other measurable parameters relevant to the medical condition being monitored.

Also disclosed herein is a method of monitoring treatment and providing care to a patient suffering from a chronic disease or condition, comprising:
providing a medical device for administering a treatment drug to the patient,
providing a computerized patient care system comprising a server system configured to receive and transmit data via a communications network to and from users including patients and health care professionals, the server system comprising a database configured to store data related to the patient, an application server including patient care software components for neurodegenerative disease management and patient information management, a communication server for data transfer through the communications network,
processing and storing data related to patient care on the server system,
transferring medical device usage data comprising data on the usage of said medical device through the communications network to the server system, and
processing said medical device usage data in conjunction with patient data to generate a report or a plurality of reports related to the treatment of the patient,
providing remote access to the reports via the communications network to registered users of the patient care system as a function of respective roles and privileges of the registered users stored in the server system.

The method may advantageously comprise:
providing a client side software application installable on a mobile user interface device (UID), such as a phone or computer tablet,
uploading medical device usage data to the patient information management program application via the WTT remote service data upload application on the server system.

The method may advantageously comprise:
uploading medical device usage data directly from the medical device to the patient information management program application via a WTT remote service data upload application on the server system.

The method may advantageously comprise:
generating a report by the reports component software, in a form selected from any one or more of tables, charts, lists, diagrams or graphical representations, said report based on information selected from any one or more of drug administration history, adherence to treatment regimen data, patient outcome reports, patient health reports, patient physiological data reports, medical device settings, treatment regimen data, and any combination of aforesaid information.

The method may advantageously comprise:
generating a composite report made of two or more reports for simultaneous display on a user interface device (UID) display, including composite adherence to treatment regimen and patient outcome reports to facilitate evaluation of the effects of non-adherence to the treatment regimen.

The adherence to the treatment regimen may advantageously be represented by a first graphical representation, and the patient outcome report relates to a physiological state of a patient and is represented by a second graphical representation, the first and second graphical representations having a common time scale such that an operator can compare the adherence to the treatment regimen and the physiological state of the patient.

Alternatively, the composite adherence report is displayed on a first time scale and the patient outcome report is displayed on a second time scale, wherein the first and second time scale are aligned and displayed simultaneously such that the composite adherence report and the patient outcome report are displayed on a common time scale, such that an operator can compare and/or correlate the adherence to the patient outcome report over a period of time.

Alternatively, the composite adherence report is displayed on a first time scale and the patient outcome report is displayed on a second time scale, wherein the first and second time scale are displaced in relation to each other, the patient outcome report being displayed on a common time scale with the composite adherence report and, such that an operator can compare and/or correlate the adherence to the patient outcome report over a period of time. An advantage is that for some health effects, there is a delay between the medication taken or missed and a measurable or perceived outcome effect of the patient.

The time scale may display adherence data and patient outcome reports per hour, day, week, month, year or any combination thereof.

The method may advantageously comprise:
transmitting by means of the notification services software component, email and/or SMS (Short Message Service) notifications to patients and optionally other users of the system, notifications selected from a group including alarms, visit reminders, treatment regime information, health information, drug information.

The method may advantageously comprise:
providing to patients online access to the tests,
automatically capturing online results of said tests by the patient therapy software and/or hardware components,
feeding said test results into the reports software component and/or into the database.

The method may advantageously comprise:
providing to patients online access to the training exercises selected from any one or more of cognition training, mobility training, speech training, vision training, cardiovascular exercise, physiotherapy.

The method may advantageously comprise:
receiving data from sensors relating to physiological measurements of the patient's measured physiological data;
transmitting said data from sensors to the server system via a communications equipped device selected from the patient's user interface device and/or medical device and/or by the sensing or training device, wherein physiological data is selected from any one or more of body temperature, blood pressure, pulse rate, galvanic skin response, surface electromyography, electroencephalography measurements, oculography measurements, electrocardiography measurements, breathing sensor measurements, blood sugar sensor measurements.

According to an aspect of the invention, also disclosed herein is a method for evaluating the efficacy of a treatment regimen for a chronic condition or disease comprising:
providing a medical device to deliver a medicament,
transmitting usage data of the medical device to a computing system
calculating adherence to a treatment regimen based on a prescribed treatment regimen data and said usage data,
transmitting to the computing system a patient reported outcome
generating in the computing system a report comprising a first graphical representation of the adherence to a treatment regimen and a second graphical representation of the patient reported outcome, said first and second graphical representations comprising a common time scale
rendering said report accessible to a health care professional for display on a screen of a user interface device, wherein said first and second graphical representations are simultaneously displayed.

An advantage of measuring the patient reported outcome is that a more complete picture of the patient's experience of life with diseases such as multiple sclerosis can be obtained, in view of the patient's perception of the effects of treatment and the disease course, thereby providing a quantifiable and broader measure of the impact of disease.

The computing system may advantageously comprise one or more of the features of a server system of a patient care system as described hereinabove.

In a particular aspect of the invention, the chronic condition or disease is a neurodegenerative disease including Multiple Sclerosis.

In a particular aspect of the invention, the chronic condition or disease is growth hormone deficiency.

Also disclosed herein is a patient care system comprising: a medical device to deliver a medicament; a first data input device operable to acquire first data, said first data relating to the use of the medical device; a second data input device operable to acquire second data, said second data relating to a physiological state of a patient; at least one processing unit operable to communicate with the first and second data input device to acquire the first and second data, and operable to process the first data to create processed first data, wherein the processed first data relates to adherence to the treatment regimen; and a user interface device comprising a display unit operable to communicate with the processing unit and to display a first graphical representation representing adherence to the treatment regimen and a second graphical representation representing said second data, such that an operator can compare the adherence to the treatment regimen and the second data, wherein said second data comprises data collected from the patient in the form of responses to questions in a clinically validated questionnaire.

In an embodiment, the invention provides a computerised medical system comprising: a medical device for administering a medical treatment to a patient, a server system, a transmitter arranged to communicate with the medical device and to transmit first data to the server, said first data relating to the use of the medical device, a first computer terminal arranged to transmit second data to the server, said second data relating to the health of the patient, the server being arranged to store said first and second data, and a second computer terminal arranged to communicate with the server system and to simultaneously display a first graphical representation representing an adherence to the medical treatment deduced from said first data and a second graphical representation representing said second data.

Also disclosed herein in relation to an embodiment of the invention is a method to monitor the efficacy of a treatment regimen comprising: transmitting first data to a processing unit, said first data relating to the use of a medical device; transmitting second data to a processing unit, said second data relating to a physiological state of a patient; processing data to create processed first data, wherein the processed first data relates to adherence to the treatment regimen; and displaying a first graph representing adherence to the treatment regimen and a second graph representing said second data on a display unit such that an operator can compare the adherence to the treatment regimen and the second data.

The processing unit may be in a remotely accessible server system.

The first data may include data selected from: the type of medication administered, the dose of medication administered, the mode of administration, the time of administration, the date of administration and the frequency of administration. A processing unit may be provided to process the first data between its acquisition by the first data input device and its display by the display unit of a user interface device to represent adherence to a pre-determined treatment regimen. The second data may include data selected from: patient reported outcomes (PROs), health test results and physiological data.

Preferably, the system is operable to time stamp the first data by means of a clock module such that the time and/or date of use of the medical device is recorded. Preferably, the system is operable to time stamp the second data by means of a clock module such that the time and/or date of the physiological state of the patient is recorded. Ideally, both the first data and the second data are time stamped. In a preferred embodiment, the first data input device time stamps the first data and the second data input device time stamps the second data. The term “time stamped” refers to logging the time and/or date that the data was input. For example, the first data input device may be operable to log the time and/or date of each medical administration. Optionally, the second data input device may be operable to log the time and/or date of each patient reported outcome (PRO), health test result and physiological data.

Preferably the display unit is operable to display the first graphical representation and the second graphical representation on a common time axis. A time axis is a plot of time. Advantageously, this enables a user, in particular a health care professional, to determine whether or not there is a correlation between the processed first data and the second data over a defined time course. The correlation can be used to monitor the efficacy of a treatment regimen, such as a dosing regimen. For example, the user may be able to determine whether or not adherence to a treatment regimen has a positive effect on a physiological state of a patient. In certain embodiments, the treatment regimen is pre-determined. Preferably, the user interface device (UID) is operable to display on the display unit the first graphical representation and second graphical representation simultaneously.

Preferably, the user interface device display unit is operable to superimpose the first graphical representation and the second graphical representation. Advantageously, this enables the operator to view the first and second graphical representations together. As such, a possible correlation between the first and second graphical representations can be observed quickly and clearly.

Preferably, the UID display unit is operable to display the processed first data as a first graphical representation. Preferably, the display unit is operable to display the second data as second graphical representation. Advantageously, this enables the operator to observe changes in the processed first data and/or changes in the second data. Optionally, the processed first and second data is time stamped. In this embodiment, the processed first and second data can be plotted on a graph having a time axis. Ideally, the processed first and second data is plotted on a corresponding time axis.

The first graphical representation may be superimposed in front of or behind the second graphical representation.

The user interface device on which the graphical representations are displayed may be physically and geographically separated from other devices selected from: the processing unit, the medical device; the first data input device and the second data input device. The term “physical separation” means that no physical contact occurs between one or more items. The term “geographical separation” means that one or more items are located in different geographical places, such as different buildings, places, villages, towns, cities or counties. In general, the terms “separate” and “separated” refer to physical separation and optionally geographical separation. Physically and geographically separating the display unit from the above mentioned one or more devices is advantageous because it allows the data to be reviewed by an operator who is not the patient, wherein the operator is geographically separated from the patient. The operator may be a care giver, such as a healthcare practitioner or a field nurse or a healthcare payer, e.g. a health care insurance company. The healthcare practitioner may be a doctor or a nurse.

The UID with display unit may be a mobile phone (e.g. a smart phone) or a computer (e.g. a digital tablet or a PC) that is operated by the care giver. Typically a field nurse may compare the first and second data on a mobile phone. Typically the doctor may compare the processed first and second data on a PC in a doctor's surgery. Typically the health care insurance company may compare the processed first and second data on a PC in an insurance office. By comparing the processed first and second data, the care giver may determine whether or not there is a correlation between the processed first data and the second data. The correlation can be used to monitor the efficacy of a treatment regimen. The operator of the display unit may also be the patient. In this embodiment the display unit may be physically separated from devices selected from: the processing unit, the medical device; the first data input device and the second data input device. Alternatively, the display unit, the first data input device and the second data input device may be one and a same device. In a further variant of this embodiment, the display unit, the medical device, the first data input device and the second data input device may be one and a same device. Advantageously, the patient can compare the processed first and second data on a display unit, wherein the display unit is selected from a mobile phone (such as a smart phone) or a computer, such as a PC or a digital tablet. By comparing the processed first and second data, the patient may determine whether or not there is a correlation between the processed first data and the second data. The correlation can be used to monitor the efficacy of a treatment regimen.

The processing unit may be physically and geographically separated from other hardware components of the system, including the medical device, the first data input device, the second data input device and the user interface device comprising a display unit. Advantageously, this allows the first and second data to be stored and/or processed on the processing unit in a secure location. This improves the security of the data. In a preferred embodiment, the processing unit is in a server system.

Preferably the first data, processed first data and second data is encrypted. Advantageously, this improves the security of the data.

In an embodiment, the processing unit is operable to communicate with the first data input device and/or second data input device and/or the UID with display unit via one or more medical device connection stations comprising wireless telecommunication transfer technology (WTT). In an embodiment the medical device connection station comprises a subscriber identity module card (sim card) for connection to a mobile phone network, such as the GSM (Global System for Module Communication) or the UMTS (Universal Mobile Telecommunications System).

In an embodiment the medical device connection station can be physically separated from other devices selected from: the processing unit, the medical device, the first data input device, the second data input device and the UID with display unit. The medical device connection station may be a base onto which the medical device can dock.

The medical device connection station may communicate with the medical device via infrared, radiofrequency or electrical communication. As such, the first and/or second data can be communicated from the medical device to the medical device connection station via infrared, radiofrequency or electrical communication. In an advantageous embodiment, the medical device connection station is a WTT transmitter enabling transmission of data to the processing unit via a mobile phone communications network.

In an embodiment, the medical device comprises one of the at least one processing unit operable to generate the first and second graphical representations.

In an embodiment, the medical device connection station connects to a user interface device in the form of a computer, such as a patient's PC or digital tablet. The connection may be wireless or it may be a wired connection, such as a USB connection. The transmitter may transmit the first and/or second data to this computer. The computer may then transmit the first and/or second data to the processing unit. The computer may transmit the data via an internet connection.

In an embodiment, the first data input device is incorporated in the medical device, whereby the medical device can acquire the first data. The medical device can record data selected from: type of medication administered, dose of medication administered, mode of administration, time of administration, date of administration and frequency of administration, and optionally compute therefrom adherence to a pre-determined treatment regimen. The medical device may communicate with the processing unit in the server system via a medical device connection station. Alternatively, the medical device may incorporate a wireless transmitter, in particular a WTT transmitter.

The second data input device may be a mobile phone (e.g. a smart phone) or a computer, such as a PC or a digital tablet. A patient can input the second data to into their mobile phone or computer, wherein the second data relates to a physiological state of the patient. In this embodiment, the medical device is physically separate from the second data input device (e.g. the mobile phone or computer). Advantageously, by using a mobile phone or a computer as a second data input device, the medical device requires less hardware and is therefore lighter and smaller as compared to if the medical device and the second data input device were one and a same device.

In an alternative embodiment, the first data input device, the second data input device and, optionally, the transmitter are incorporated in a single device. Preferably, this single device is a mobile phone (e.g. a smart phone) or computer. The computer may be a PC or a digital tablet. In this embodiment, the medical device is physically separate from the first data input device, the second data input device and the transmitter (e.g. the mobile phone or computer). Advantageously, this allows for the medical device to be more easily disposable, for instance for single-use administration. Advantageously, the first and second data can be input into the same user interface device.

In an embodiment, the user interface device, such as a mobile phone or computer, is configured to communicate with the medical device, such as a disposable single-use medical device, via a label borne by the medical device. Ideally, the label is a Near Field Communication (NFC) chip and/or a Quick Response (QR) code. The medical device can be brought into contact or near contact with the mobile phone or computer equipped with an NFC transceiver, respectively QR code reader. This would trigger identification of the label by the phone or computer. Advantageously, the mobile phone or computer is operable to identify an individual medical device. Thus, the mobile phone or computer may acquire data selected from: the first data, the second data and data identifying the medical device and may transmit this data to the server system.

In an alternative embodiment the medical device, the first data input device and the second data input device are incorporated in a same device. As such, the medical device may be operable to acquire the first data and the second data. Advantageously, the patient need only input data into a single device. This would be beneficial to patients who do not wish to use a mobile phone or a computer. The medical device may also comprise a transmitter, whereby the medical device is operable to transmit the first data and second data to the processing unit physically or geographically separated from the medical device. Alternatively, the transmitter may be a base onto which the medical device can dock. The transmitter may advantageously be a WTT transmitter. Further optionally, additional second data, such as health test results, may be acquired by a further second data input device, such as a computer belonging to the patient.

The medical device may be a device for delivering medicament to a patient. The medical device may be an injection device, such as a subcutaneous injection device, an intravenous injection device or an intramuscular injection device. The medical device may be an electronic medical device or a mechanical device. Preferably, the medical device is an electronic subcutaneous injection device.

Alternatively, the medical device may be a pill dispenser, an inhaler or a topical administrator, such as a spray dispenser.

In a particular aspect of the invention, the patient care system, the method for monitoring the efficacy of a treatment regimen or monitoring treatment is configured for a disease selected from the group of multiple sclerosis, growth hormone deficiency, rheumatoid arthritis, juvenile rheumatoid arthritis, psoriasis, plaque psoriasis, Crohn's disease, juvenile Crohn's disease, asthma, psoriatic arthritis, ulcerative colitis, systemic lupus erythematosus, ankylosing spondylitis and breast cancer.

In a particular aspect of the invention, the patient care system, the method for monitoring the efficacy of a treatment regimen or monitoring treatment is configured for a patient suffering from cognitive depression.

In a particular aspect of the invention, the patient care system, the method for monitoring the efficacy of a treatment regimen or monitoring treatment is configured for a patient suffering from fatigue.

The term “data” is used to describe the first data and/or the second data and/or processed first data. Optionally, the term “data” may also be used to describe the third data and/or the fourth data, which are further defined below.

### The First Data

In an embodiment, the first data relates to data selected from: type of medication administered, dose of medication administered, mode of administration, time of administration, date of administration and frequency of administration.

In a preferred embodiment of the invention, the system comprises a processing unit that is operable to process the first data. In a preferred embodiment of the invention, the method further comprises processing the first data to produce processed first data. The processed first data relates to adherence to a pre-determined treatment regimen.

The system or method of the invention may further comprise third data. Said third data comprises a pre-determined treatment regimen. The third data may comprise data selected from: pre-determined type of medication, pre-determined dose of medication, pre-determined type of administration, pre-determined time of administration, pre-determined date of administration and pre-determined frequency of administrations.

The processed first data may comprise data relating to adherence to a pre-determined treatment regimen, this may be referred to as treatment regimen adherence data. The treatment regimen adherence data is a correlation between pre-determined treatment regimen data and actual treatment regimen data. The pre-determined treatment regimen may be pre-determined by a care giver, such as a doctor. In an embodiment, the display unit displays the processed first data (treatment regimen adherence data) and the second data (a physiological state of the patient).

Thus, the processing unit is operable to calculate the adherence to a pre-determined treatment regimen by processing the first data. The processed first data is calculated by comparing the first data to the third data.

In other words, the adherence to a pre-determined treatment regimen is calculated by comparing the actual treatment regimen data to the pre-determined treatment regimen data.

A pre-determined treatment regimen may be referred to as a prescribed treatment regimen.

Thus, an adherence of 100% indicates that the patient has completely adhered to the pre-determined treatment regimen, while an adherence lower than 100% indicates that the patient did not completely adhere to the pre-determined treatment regimen, for example the patient may not have injected all prescribed doses.

The UID display unit may display the processed first data as a percentage of adherence to the third data (i.e. the pre-determined treatment regimen). The percentages may be displayed as a graph. Thus, in one embodiment, the display unit displays a processed first data graph.

Preferably, the UID display unit is operable to display the processed first data and the second data graphically on a common time axis. Advantageously, this enables the operator to determine whether or not there is a correlation between the adherence to a pre-determined treatment regimen and a physiological state of a patient. Ideally, the UID display unit is operable to display the processed first data and the second data simultaneously, such as side by side or superimposed upon each other.

The pre-determined treatment regimen (i.e. the third data) may comprise data selected from: pre-determined type of medication, pre-determined dose of medication, pre-determined mode of administration, pre-determined time of administration, pre-determined date of administration and pre-determined frequency of administration. Thus, the pre-determined treatment regimen data contains time and/or date information.

The actual treatment regimen data (i.e. first data) may comprise data selected from: type of medication administered, dose of medication administered, type of administration, time of administration, date of administration of medication and frequency of administration of medication. The actual treatment regimen data (i.e. first data) may be time stamped, e.g. by a clock module. Processing of this time stamped first data results in time stamped processed first data.

The processing unit is operable to compare the time stamped first data to the time/date information contained within the third data.

The mode of administration may be injection such as subcutaneous injection, intravenous injection, intramuscular injection, preferably the mode of administration is subcutaneous injection. In an alternative embodiment, the mode of administration is oral or topical administration.

The first data input device may be a UID in the form of a mobile phone comprising a smart phone application or a computer comprising a web based application, wherein the application is operable to acquire the first data.

A dosing regimen is a form of treatment regimen.

### The Second Data

In an embodiment, the second data comprises data selected from: patient reported outcomes (PROs), health test results and physiological data. The second data may advantageously comprise a PRO.

Patient reported outcomes (PROs) are responses to a clinically validated questionnaire. The questionnaire may contain questions permitting evaluating the level of criteria selected from general health, pain, cognition, fatigue, bladder condition, bowel condition, sexual satisfaction, visual impairment, mental health, feelings and emotions, depression, sleep and other health criteria of the patient. The questionnaire may require the patient to score one or more of the criteria on a sliding scale.

Health test include mobility tests, walking tests, vision tests and/or cognition tests. The tests may be done by the patient at home using a computer and test apparatuses such as motion sensors connected to the computer, e.g. a radiofrequency connection (e.g. Bluetooth® or Wi-Fi).

Physiological data includes, but is not limited to, body temperature, blood pressure, heart rate, galvanic skin response activity, breathing activity, blood sugar level, brain activity, eye movement activity, muscle movement activity, and height (in the case of a growth hormone treatment) of the patient. The system may comprise a height measuring device.

The second data is objective.

The second data input device may be a UID in the form of a mobile phone comprising a smart phone application or a computer comprising a web based application, wherein the application is operable to acquire the second data.

### The Third Data

The medical device, computer, mobile phone or server may comprise third data. Said third data comprises a pre-determined treatment regimen. The third data may comprise data selected from: pre-determined type of medication, pre-determined dose of medication, pre-determined type of administration, pre-determined time of administration, pre-determined date of administration and pre-determined frequency of administrations. Advantageously, the medical device, computer or mobile phone may remind or instruct the user to carry out the pre-determined treatment regimen.

### The Fourth Data

Fourth data may also be entered into the medical device or UID, for instance a computer or mobile phone. Said fourth data comprises a date and time of an appointment with a care giver. Advantageously, the medical device or UID may be operable to provide an alert to remind a patient of their appointment.

### Data Input

The first data may be manually input into the first data input device. For example, the first data input device may have a user interface such as a touch screen, a keyboard or a computer mouse. The patient may input the data into the first data input device via the user interface. Preferably, the user interface is a touch screen. Ideally, the first data input device is a medical device or a mobile phone comprising a touch screen.

The first data may be detected by the first data input device. For example, the first data input device may be a medical device that is operable to automatically acquire data selected from: type of medication administered, dose of medication administered, mode of administration, time of administration, date of administration and frequency of administration.

The second data may be manually input into the second data input device. For example, the second data input device may have a user interface such as a touch screen, a keyboard or a computer mouse. The patient may input the data into the second data input device via the user interface. Preferably, the user interface is a touch screen. Ideally, the second data input device is a medical device or a mobile phone having a touch screen.

The second data may be detected by the second data input device, e.g. by a sensor. The sensor may be selected from: an electroencephalography (EEG) sensor, an electrocardiography (ECG or EKG) sensor, a skin surface sensor, an electro- or video-oculography sensor, breathing sensor, blood sugar sensor, a motion sensor, a vision sensor and a height sensor. The sensor may transmit data to the second data input device via a radiofrequency connection (e.g. Bluetooth® or Wifi). The second data may be input into multiple second data input devices.

### Data Storage and Processing

In a preferred embodiment, the system includes a data processing unit for processing the first data and/or the second data incorporated in a remotely accessible server system. The server system is operable to produce the first and second graphical representations and transmit them to the display unit.

In a preferred embodiment, the server system includes a database for storing the first data, processed first data and/or the second data.

In an embodiment, the server system is physically and geographically separated from the medical device and user interface devices (UID) of the system operating as the first data input device, the second data input device and the display unit. Advantageously, this allows the data to be stored and/or processed on the server in a secure location, the extent and type of information accessible by different users depending on their roles and functions. This improves global accessibility as well as the security of the data, and furthermore allows collection of data over time that may be used to gain a better understanding of the effects of treatment on the disease.

In an embodiment, the medical device comprises a data storing module operable to store the first and/or second data offline, which may then be transmitted to the server system at a time that is convenient to the operator of the medical device. Thus, data that has been collected over a period of time, such as days weeks or months, e.g. up to twelve months, can be transmitted simultaneously to the server system.

### Display of Data

The UID display unit is operable to display the processed first data and second data to enable an operator to compare the processed first data and second data. Advantageously, this enables an operator to more conveniently determine whether there is a correlation between the processed first data and second data.

In a preferred embodiment, the processed first data can be displayed as a diagram, preferably a first graph. Likewise, the second data can be displayed as a diagram, preferably a second graph. Advantageously the first and second graphs can be displayed simultaneously. For example the first and second graphs can be displayed next to each other or superimposed over one another. Alternatively, the processed first data and second data can be displayed in the form of a calendar, such as a daily, weekly or monthly calendar.

### Telecommunication Networks

Data is transmitted within the system via telecommunication networks selected from: a wireless telecommunication technology (WTT) network, also known as a mobile or cellular phone network, a fixed line telephone network, an Internet network and a local computer network such as an intranet network including various communication protocols and means such as WiFi and Ethernet.

In many embodiments, data can be transmitted over one or more of these networks. For example, the first data input device may transmit first data to a computer via a mobile phone network. The computer may then transfer the data to the server system by an Internet network. For example, the second data input device may transmit second data to a server via a mobile phone network. For example, the server system may transmit processed first data and second data to a display unit via an Internet network.

Advantageously, the transmission of data between devices may be wireless.

### Medical Devices

The medical device is for administering medical treatment to a patient, in particular for administering a medical treatment drug. In one aspect of the invention, the medical treatment is treatment for multiple sclerosis, such as interferon beta-1a, e.g. Rebif® or Avonex®. In another aspect of the invention, the medical treatment is treatment for growth hormone deficiency, such as recombinant growth hormone, e.g. Saizen®.

In an embodiment, the medical device is an injection device, such as a subcutaneous injection device, an intravenous injection device or an intramuscular injection device.

Preferably, the device is an electronic subcutaneous injection device, for instance of a type commercially known as Rebismart®, Rebidose®, or Easypod®. Rebismart® administers Rebif® for treatment of multiple sclerosis. Rebidose® is a disposable medical device which administers Rebif® for treatment of multiple sclerosis. Easypod® administers Saizen® for treatment of growth hormone deficiency.

In an embodiment, the medical device for multiple sclerosis treatment incorporates the first data input device. Advantageously, this means that the medical device for multiple sclerosis treatment can acquire the first data. In this embodiment, the medical device for multiple sclerosis treatment can record data selected from: adherence to a pre-determined treatment regimen; type of medication administered, dose of medication administered, mode of administration, time of administration, date of administration and frequency of administration.

The medical device for multiple sclerosis treatment may communicate with a server via a transmitter.

Preferably, the transmitter is a base to which the medical device for multiple sclerosis treatment is docked. The medical device for multiple sclerosis treatment may be operable to communicate with the transmitter by infrared, radiofrequency or electrical connection. Ideally, the transmitter is a wireless transmitter.

The medical device for growth hormone deficiency treatment may operate in the same way as the medical device for multiple sclerosis treatment.

The medical device for multiple sclerosis or growth hormone deficiency treatment may be used in conjunction with a second data input device. The second data input device may be a mobile phone or a computer, such as a PC or a digital tablet comprising a client side software application for capture and processing of the data. In this embodiment, a second transmitter and the mobile phone or computer are incorporated in a same device. As such, a patient can input the second data to into their mobile phone or computer, wherein the second data relates to a physiological state of the patient. In this embodiment, the medical device for multiple sclerosis or growth hormone deficiency treatment is physically separate from the second data input device (e.g. the mobile phone or computer). Advantageously, by using a mobile phone or a computer as a second data input device, it means that the medical device for multiple sclerosis or growth hormone deficiency treatment requires less hardware and are therefore lighter and smaller than as compared to if the medical device and the second data input device were one and a same device.

In an alternative embodiment, the medical device for multiple sclerosis or growth hormone deficiency treatment is disposable and physically separate from the first data input device, the second data input device and the transmitter. In this embodiment, the first data input device, the second data input device and the transmitter may be incorporated in a same device, such as a mobile phone or a computer comprising a client side software application for capture and processing of the data. The first and second data can be input into the same mobile phone or computer which improves convenience for the user.

Preferably, the mobile phone or computer is arranged to communicate with the disposable medical device for multiple sclerosis or growth hormone deficiency treatment via a label borne by the disposable medical device. Ideally, the label is a Near Field Communication chip and/or a Quick Response code. The disposable medical device can be brought into contact or near contact with the UID in the form of a mobile phone or computer. This would trigger identification of the label by the phone or computer. Advantageously, the mobile phone or computer comprises a client side software application operable to identify an individual the disposable medical device. Thus, the mobile phone or computer may acquire: the first data, the second data and data identifying the medical device, and then transmit this data to the server system.

Other features and advantages of the present invention will become apparent from the reading of the following detailed description made with reference to the appended drawings.

### BRIEF DESCRIPTION OF THE DRAWINGS

FIG. 1 is a schematic overview illustration of a patient care system according to an embodiment of the invention;

FIG. 2 is a schematic overview illustration of a server system of a patient care system according to an embodiment of the invention;

FIG. 3 is a schematic overview illustration of a functional architecture of a server system of a patient care system according to an embodiment of the invention;

FIG. 4 is a schematic overview illustration of a software system context diagram of a server system of a patient care system according to an embodiment of the invention;

FIG. 5 is a schematic overview chart of software components of a patient care system according to an embodiment of the invention;

FIG. 6 shows a screen interface illustrating a composite report that a physician may use to analyse patient adherence to treatment, according to an embodiment of the invention

FIGS. 7a to 7c are illustrations of medical devices according to embodiments of the invention, FIG. 7a illustrating a reusable medical device docked on a medical device connection station and FIG. 7b illustrates a single-use disposable medical device;

FIGS. 8a to 8c are simplified schematic block diagrams illustrating an aspect of an embodiment of a patient care system according to the invention.

### DETAILED DESCRIPTION OF EXEMPLARY EMBODIMENTS

In the following description, in exemplary embodiments of the present invention the patient care system and method for monitoring or evaluating the efficacy of treatment are configured for treatment of multiple sclerosis or growth hormone deficiency. The present invention may also be applied in treatments for other diseases such as rheumatoid arthritis, juvenile rheumatoid arthritis, psoriasis, plaque psoriasis, Crohn's disease, juvenile Crohn's disease, asthma, psoriatic arthritis, ulcerative colitis, systemic lupus erythematosus, ankylosing spondylitis and breast cancer.

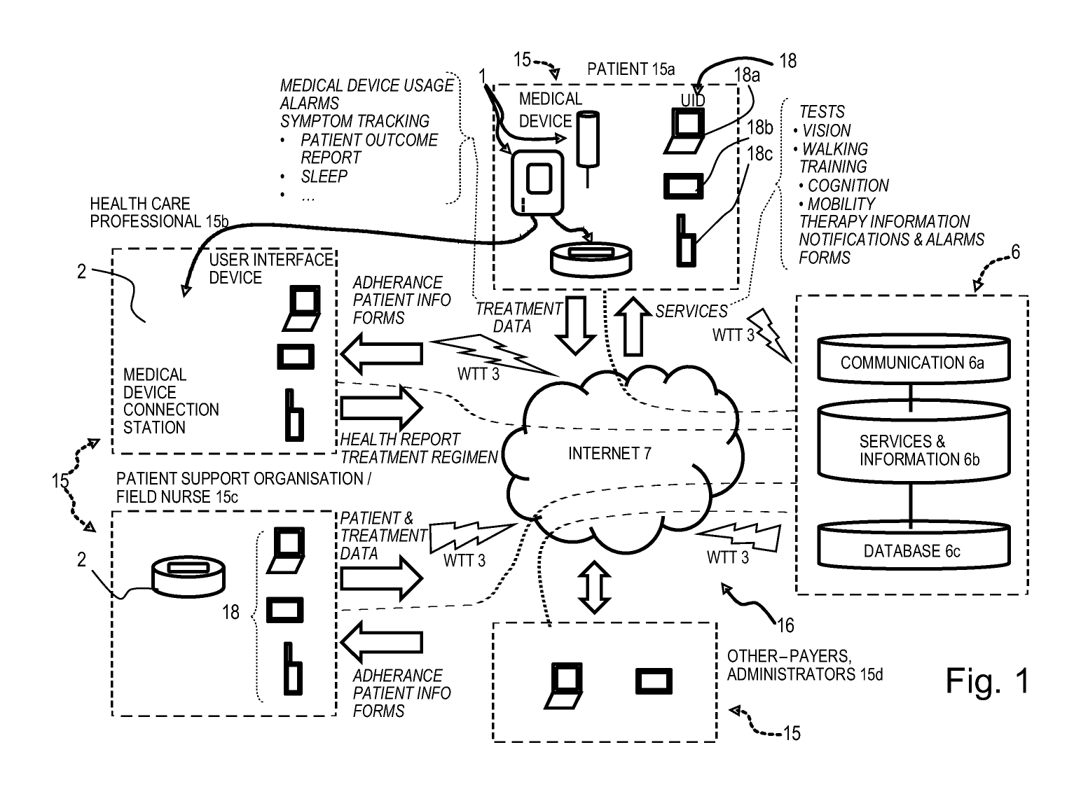

Referring to FIG. 1, a patient care system according to the invention comprises a medical device 1 for administering a medical treatment to a patient, a server system 6, a user interface device 18, and depending on the embodiment, a medical device connection station 2. The server system 6 is configured to process and/or store information related to patient care and accessory services, such information being received and transmitted via a communications network 16, in particular a global computer network such as the internet 7. The communications network may further include a wireless telecommunications transfer (WTT) network, for instance a mobile phone network 3, and direct point-to-point communication between users 15 and the server system 6.

Users include patients and health care professionals (HCP), in particular physicians and nurses. Users may further include patient support organisations or persons (PSO), for instance including field nurses. Other examples of support organisations may include local support groups, similar patient communities and information about locations for medication supply. Users may further include one or more of system administrators, health insurance organisations, suppliers of health services, manufacturers and suppliers of medication, pharmacies, and payers of health services. HCPs manage the patient related information in the system. This role creates patient entries, and manages and monitors the patients' related data in the system to enable better treatment and care to the patients. A system administrator is responsible for managing the system related functions in the applications that includes creating users, roles and functionalities, and other master data. The role of a field nurse may be similar to that of the HCP and can manage all patient data. The field nurse may typically access the application using a mobile user interface device, such as a computer tablet or smartphone. A field nurse may for instance schedule appointments, meet the patients, and collect information from the patient.

User interface devices may comprise a display and means to communicate with the server system 6 or the medical device connection station, and may include personal computers 18a, computer tablets 18b, mobile phone devices 18c or other electronic computing devices with a graphical user interface and means to communicate with the server system 6 or the medical device connection station.

A user interface device may be integrated in the medical device 1 such that, depending on the embodiment, the patient does not require a separate user interface device to connect to the server system or the medical device connection station. Client application programs may be installed on the medical device and/or the user interface device, the client application program configured to establish connection with the server system 6 or medical device connection station 2 and to manage the transmittal and reception of data to, respectively from, the server system or medical device connection station.

The medical device connection station 2 may have different configurations depending on the variant. In a variant, the medical device connection station 2 may be configured as a communication interface device that serves to communicate information to and from the medical device to a user interface device, for instance the user interface device of a health care professional 15b, and/or through the communications network 16 to and from the server system 6. The medical device connection station may, in another variant, further comprise a processor and software configured to process raw data from the medical device in an information relevant form to a user 15 via the user interface device, or to the server system 6. The medical device connection station comprises a connection interface complementary to a connection interface of the medical device. The connection interface may include direct electrical contact, for instance a USB connection, or various wireless communication systems, in particular near field communication systems, which may operate with various known communication protocols such as infrared, Bluetooth, Zigbee and other radio frequency (RF) communication protocols.

Within the scope of this invention, medical devices include reusable injection devices, disposable injection devices, implantable drug delivery devices, intradermal disposable devices, and electronically readable packaging of solid form drugs, for instance pills for oral delivery, or suppositories, in electronic container or blister packaging which senses when a drug dose is removed from the container or packaging.

Data transmission between the server system 6 and users 15, via their medical device 1, and/or medical device connection station 2, and/or user interface device 18 is preferably encrypted, for instance using a public key/private key pair encryption means, a symmetric encryption means, or other encryption means known per se.

The data are stored confidentially in the server system 6 and processed and classified so as to be easily and securely accessible to the various users 15a, 15b, 15c, 15d as a function of the access rights allocated to each type of user and the identity of the user. Medical information on the patient is accessible to the patient 15a and the physician 15b, possibly also to authorized members of the patient support organisation 15c, such as field nurses, but not to other users.

The server system 6 may comprise a plurality of physical and/or virtualised servers in a single location or in a plurality of locations distributed in the communications network 16. The meaning of “server” thus encompasses hardware servers with server software dependant on the hardware server configuration, and virtualised servers installed on one or more hardware server components that are independent of the hardware server configuration. A server may also be formed by a plurality of server hardware and/or software components that are distributed in a computer network.

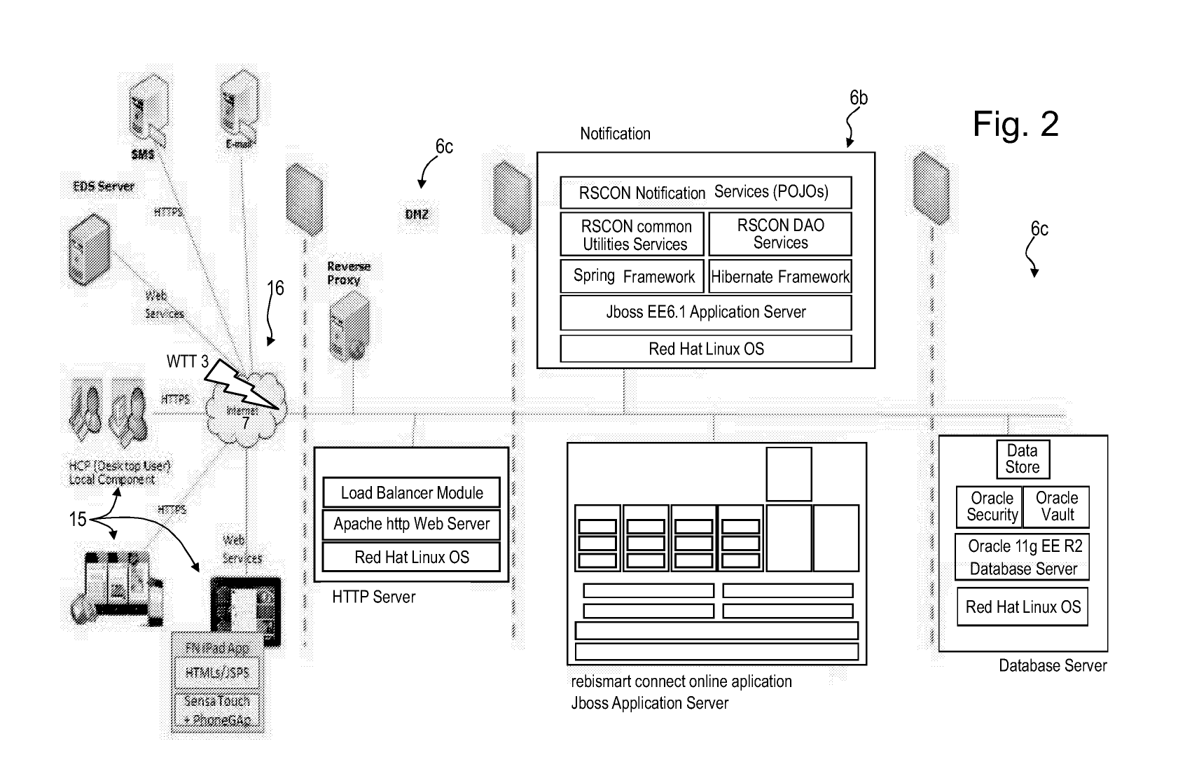

Referring to FIGS. 1 and 2, in an advantageous embodiment, the server system comprises a communications server system 6a including a web server (HTTP server), an application server system 6b, and a database 6c. The application server system may comprise an information server for handling online services accessible by the users, a notification server for handling notification services to users, a server for handling mobile components, and a server for handling data load services from local components and devices.

### Example of a Server System

In an example, a server system according to this embodiment may comprise the following features: HTTP (Hyper Text Transfer Protocol) Servers:
May be Apache HTTP Web servers in a demilitarized zone protected by a firewall that will only allow HTTPS or HTTPS SOAP requests. No other protocol is allowed to hit the HTTP servers. All requests from user devices are through SSL (secure socket layer) to ensure that all communication between the user devices and HTTP servers is encrypted during transmission.
No patient or medical data will reside on the HTTP Servers
The HTTP servers are load balanced to provide performance and high availability. If one of the Web servers goes down for any reason, the performance of the system in case of peak workloads might be impacted but the system will still be available, and able to handle user requests.

### Application Servers:

May be a JBoss Application Server clustered to provide high availability
JBoss clustering will handle session management

### Application Servers for Notification Component:

The JBoss application server hosts the notification component, for instance for handling notifications sent per email or SMS (short message service). This server does not need to be clustered since it will not receive requests from users.

### Application Servers for Mobile Components:

The JBoss Application Server is preferably clustered to provide high availability
   Application Servers for Data Load Services from Local Component and Devices:
The JBoss Application Server may be clustered to provide high availability

### Database:

May for instance be an Oracle™ Enterprise Edition database that is clustered to provide high availability
An Oracle Advance Security Option can be used to provide data encryption for stored data.

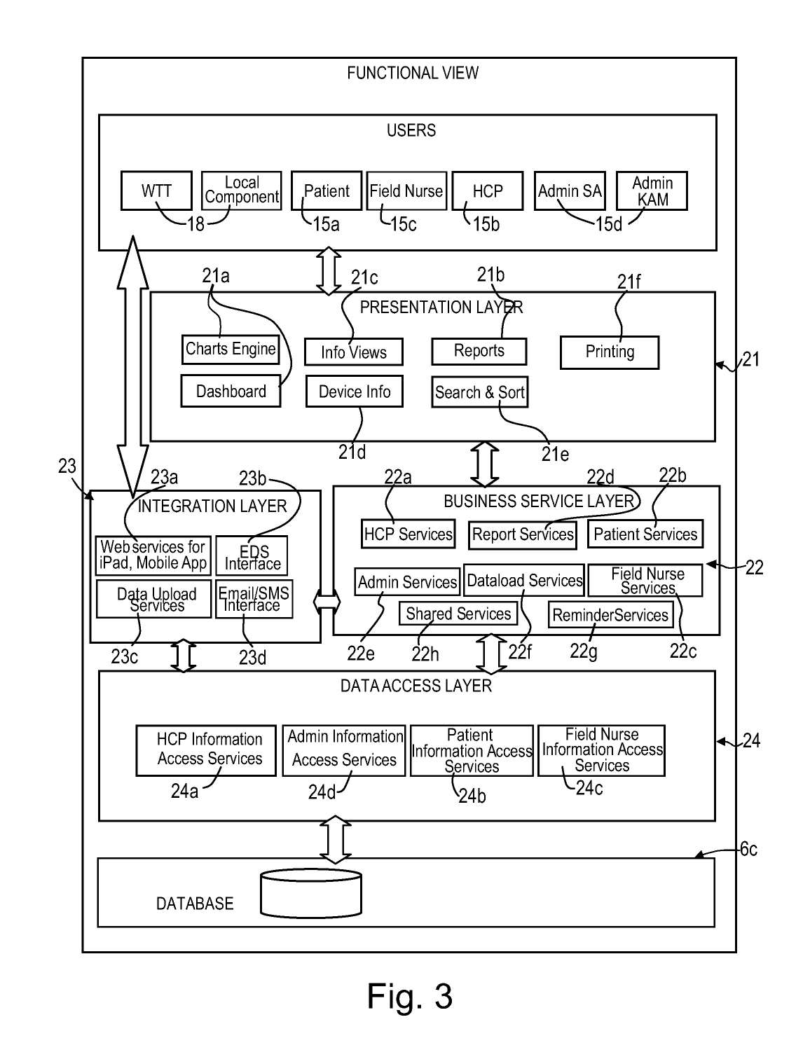

Referring to FIG. 3, a functional diagram of the program architecture of an exemplary embodiment of a computerized patient care system according to the invention is illustrated. The program functions comprise presentation program components 21, business services program components 22, integration program components 23, and data access program components 24.

The presentation components are responsible for managing the requests and response from the user's interface device, for instance from a network browser on the user's interface device, and rendering the presentation to the respective users. The presentation components are specialized to render charts 21a, reports 21b, views 21c, device information 21d, data entry and search 21e, and prints 21f.

The business services components are responsible for providing all the functionality required for the HCP and administrator users. They hold the business logic to process and provide the data back and forth to the users. The business services components are grouped based on the functionality they provide to the users, and include HCP services 22a, patient services 22b, field nurse services 22c, report generation services 22d, administrative services 22e, data load services 22f, reminder services 22g, and shared services 22h. The shared services provide functionality, such as logging, error handling, audit trail, caching, security, and notification used by all the other program modules.

Integration Components are responsible for providing the interfaces to various external interface devices, including web services for mobile applications 23a, EDS interface 23b, data upload services 23c, email and SMS interface 23d. These program components may include:
programs to upload device data from the local component and the WTT system
Web-services to provide patient and associated data to mobile applications (e.g. iPad™ application)
Web-services to provide field nurse and associated data to mobile applications (e.g. iPad™ application)
Integration with EDS (Enterprise Data Services)
E-mail gateway integration
SMS gateway integration

The data access components provide functionality to manage data storage and retrieval from the database, and include HCP information access services 24a, patient information access services 24b, field nurse information access services 24c and administrator information access services 24d.

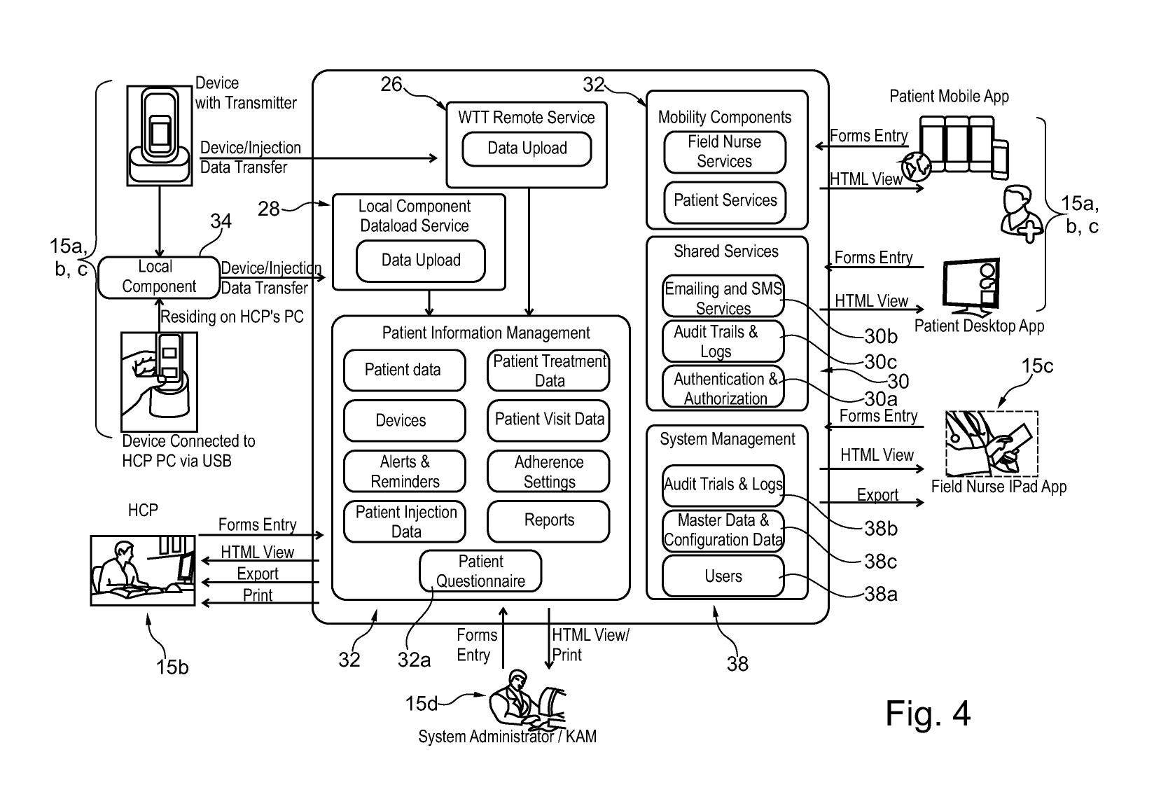

FIG. 4 illustrates an exemplary embodiment of the interactions between users and the server system on a contextual level. In this example, the various users, including patients 15a, health care professionals 15b, field nurses 15c, and administrators and key account managers (KAM) 15d access the software applications on the server system 6 remotely through the communications network 16, including in particular through the internet 7. Administrators and KAM and health care professional may typically access the system via a user interface device (UID) 18 in the form of a desktop computer for instance, whereas patients and field nurses may typically access the system through a desktop UID as well as a mobile UID in the form of a smart phone or computer tablet.

For the mobile UID's, medical device data may be uploaded into the server system 6 using a WTT remote service data upload application 26 in the server system, directly from the medical device through the wireless telecommunications network 3 using the available mobile cellular systems, for instance GPRS (general packet radio service), UMTS (Universal Mobile Telecommunications System) or others. UIDs accessing the server system through the internet 7 can also upload medical device data, in particular usage data stored in the medical device, through a local component dataload service application 28 on the server system.

Only registered users can access the server system. Users are authenticated by an authentication and authorization program application 30a forming part of a shared services section 30 available to all users. User data, including identity, type, and respective roles and privileges is stored in the server system, for instance in the database 6c, and enables users to have access to authorized functionality and data based on their respective roles and privileges, for instance patient sensitive data may be available only to HCPs and patients, whereas server system administration functions may be available only to system administrators, and forms configurations available only to key account managers and system administrators.

In the example illustrated, data may be inputted into the server system 6 in the following ways:
The medical device 1, incorporating or connected to a WTT (Wireless Telecommunication Technology) transmitter in the medical device connection station 2 for direct upload of data, in particular medical device usage data, for instance injection history data, to the patient information management program application 32 via the WTT remote service data upload application 26 on the server system. The WTT module receives the data, and after successful validation stores it in the online database 6c.
The medical device 1 device can be connected to a UID (user interface device) 18 either directly or via the medical device connection station 22, the UID comprising a local component program (a client side program) 34 for the transfer of information to the server system 6. The local component program 34 receives medical device usage data, for instance injection history data, and uploads it to the server system 6 via the local component data upload service application 28 over HTTPS (Hyper Text Transfer Protocol Secure) requests. The local component data upload service application 28 validates the uploaded data and stores it in the online database 6c. As an example, the local component can communicate with the program on the server system using a Spring™ framework http invoker feature, and the medical device communication station 2 may also communicate with the WTT remote data upload service application 26 via the Spring™ HTTP (Hyper Text Transfer Protocol) invoker.
User input: HCPs, administrators, KAM (country based Key Account Managers), patients, and field nurses can log on to the server system and input various types of data fields into the server system through various forms available in the application. For instance patient information, medical device association information, and clinic information are entered by the users in the application. Sensitive patent information is stored in the database 6c in an encrypted form and is only accessible to authorized users, which may include HCPs and field nurses.

In an exemplary embodiment, in an online mode, the communication between the UIDs 18 and the various web accessible services on the server system 6 may be secured using the HTTPS protocol. In an offline mode, the medical device comprises a database (for instance SQLite) used to store the usage data locally such that user can save the medical device usage data and other data in the database of the device when the user is offline. This can use for instance a JavaScript encryption mechanism (e.g. AES 128) for the data in the local storage. The pages are available in the UID 18 in the offline mode. The user may require a password and User ID to log on to the application in the offline mode. This password maybe used as a key for the encryption/decryption of the data.

In the exemplary embodiment illustrated, the patient information management application 32 generates information in various formats for output of the server system 6, for instance including HTML views and forms for entering data, and various list and form views displaying information to the user about patient devices.

HTML view and forms may be further categorized as follows:

Various list and form views display information related to the patient for use by the patient, HCPs and field nurses and may include:
Profile—Edit
Reminders
   Configuring Adherence Threshold Values
   Schedule Reminders
Patient Reports
   History Overview Chart—allows the HCP to view graphical representation of injection history data for a selected patient.
   Injection History List—HCP can view the injection data as list for a selected patient.
   Injection History Graph—HCP can view the graphical representation of the injection data for a selected patient.
   Injection history Calendar view—HCP can view the calendar representation of the Injection data for a selected patient.
   Adherence Data Reports
   Outcome Reports
   Combinations of the above to form composite reports, most advantageously including composite adherence and outcome reports to evaluate the effects of non-adherence to the treatment regimen
Patient's Device
   Device Settings—view of current and history of device settings
   Device Registration
   Device Assigned/Un-assigned
Calendars—For Patient and field nurses only
   View day wise reminders, appointment, events
Visit and Remarks
   Add Visit and Remarks
   Edit Visit and Remarks

Various list and form views displaying information to the user about patient devices may include:
Device Management—This functionality allows user to assign and un-assign devices to the patient.
View Active Device Settings—This view allows the user to view the settings of the device assigned to a specific patient.

Server system outputs may further include a printing function allowing users to print the viewable data and a restricted data export function, for instance:
HCPs are allowed to export the patient's injection history data and device settings from the system, for instance as a comma-separated values file (CSV).
Field nurses are allowed to download patient and appointment information on their UID 18, for instance their computer tablet 18b, for offline use during patient visits. This data is encrypted and stored in a local offline database on the UID (18).

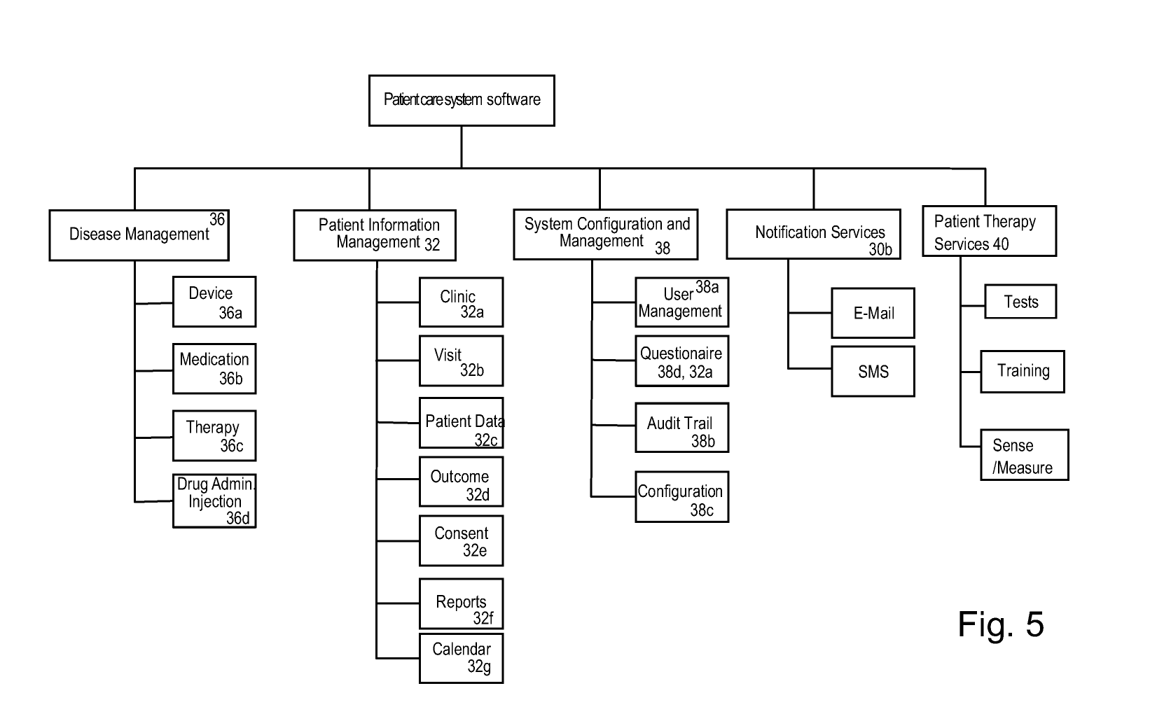

Referring to FIG. 5, in conjunction with FIG. 4, software components of the patient care system according to the invention may be categorized as follows:

i. Disease Management 36

This category comprises software components which are used to manage the disease. In a particular embodiment of the invention, the disease is a neurodegenerative disease, in particular Multiple Sclerosis. These are software components that relate to the medical device configuration and use 36a, medication 36b, therapy 36c and drug administration 36d, e.g. injection.

ii. Patient Information Management 32

This category comprises software components which are used to manage information on patients, in a particular embodiment on Multiple Sclerosis patients. These include information related to the treating clinic 32a, medical and therapeutic visits 32b, patient consents 32e, patient data 32c, treatment outcomes 32d, patient and HCP reports 32f, and calendar 32g software components.

iii. System Configuration and Management 38

This category comprises software components which are used for configuration and management of features used across the patient care system. These include user management 38a, 30a, questionnaires 38d, including patient questionnaires 32a, and audit trail software components 38b, including shared services audit trail components 30c.

iv. Notification Services 30b

This category comprises software components which are used for notification of events to users 15. These include e-mail and SMS software components 30b.

v. Patient Therapy Services 40

Patient therapy services components, also named herein patient therapy components, comprise patient therapy software and hardware components which are used to provide information to the patient inter alia to assist in improving therapy, disease management and patient reports. Patient therapy software components include online tests 40a, such as vision and walking tests, online training 40b such as cognition and mobility training, disease information for patients and patient support organisations, and online patient physiological monitoring. Online patient physiological monitoring for multiple sclerosis may comprise a digitalised version of the 9-Hole Peg Test, which is a quantitative test of upper extremity function of a patient. Patient therapy services may display instructional information in visual or audible form, for instance in the form of instruction videos and downloadable information files. Patient therapy hardware components include wearable devices for monitoring stress, sensors that detect movement during sleep, and sensors and cameras for detecting motion during the physical training.

Depending on the type of disease to be treated, additional patient therapy services components may include physiological monitoring modules for activity tracking and gait assessment, and rehabilitation, and psychological modules for stress monitoring, cognitive behavioural therapy, depression assessment and/or treatment, and fatigue monitoring and treatment.

Activity tracking may include information such as a physical exercise performed (distance walked, time and/or load), calorie consumption, and heartbeat. Gait assessment can be used as a computerised method for assessing, planning, and treating individuals with conditions affecting their ability to walk. Especially for patients having a disease which affects their ability to walk (e.g. multiple sclerosis), gait assessment can be used in order to assess, plan, and treat the disease. A gait analysis may include several cameras configured to record the position of markers attached to the patient's body. A computer calculates the trajectory of each marker in three dimensions and a model of the movement of the underlying bones can be calculated such that a complete breakdown of the movement of each joint can be determined.

E-health patient therapy services may be added to the system in order to increase the efficiency of the treatment. These e-health patient therapy services may comprise cognitive remediation (e-CR), depression and anti-fatigue treatment, rehabilitation and exercise therapy and monitoring and managing stress.

Cognitive remediation (e-CR) is a non-medicated treatment aimed at improving cognitive skills through brain training by repeated exercises. The benefits are improved cognitive skills of patients and by consequence an improved quality of life. Cognitive remediation is particularly important for neurodegenerative diseases such as multiple sclerosis (MS) as more than half of people with MS experience cognitive changes, in particular problems with learning and memory. These problems are important to address as if left untreated, the cognitive problems can affect a person's quality of life by creating limitations at work and in social interactions. Cognitive remediation can improve learning and memory, improved general satisfaction, improved planning and organizing ability, and reduced apathy.

Depression and fatigue can be treated by cognitive behavioural therapy by a specific module in the user interface device (UID) which enables an Internet accessible program for treating depression and fatigue. The system may include assessments and exercises for improving the baseline and the overall impression of well-being of the patient. The assessments may further include measuring and quantifying the depression stage by using Beck depression inventory (which is a 21-question multiple-choice self-report inventory) and/or a Multiple Sclerosis Impact Scale (which is a questionnaire with associated ratings). Depression may be assessed and/or treated by the present system and device as a secondary disease in conjunction with e.g. multiple sclerosis, or as a primary disease.

Monitoring and managing stress as a secondary disease is important in order to reduce the disease symptoms of the primary disease. For multiple sclerosis patients, it is especially important to manage the stress in order to reduce the risk of relapse and to reduce development of new brain lesions. In an embodiment, the present patient monitoring system configured for measuring stress may advantageously comprise a wearable biosensor device for measuring stress by recording for instance pulse, electrodermal activity, temperature and blood oxygenation. The biosensors may consist of a biosensor bracelet, a patch senor or any other type of suitable device configured to be in contact with the patient.

In an embodiment, the patient monitoring system may advantageously comprise an individual stress profile treatment module may also comprise a stress visualizer and event recorder. The treatment module may also be configured to provide online coaching for self-initiated intervention to manage stress.

A further therapy module may include a sleep monitoring and CBT treatment program. The program may collect measured information from a biosensor about the patient's sleep. The system is further configured to guide through the sleeping program module and assist the patient for various issues which may occur during the day or the night.

The sleeping program may also be configured to display and guide the patient through sleep improvement techniques based on CBT and optionally recommend adapted food intake or lighter drugs. Personalized Cognitive Behavioural Therapy (CBT) techniques may be configured to correct sleep schedule, thoughts, lifestyle and bedroom layout.

Rehabilitation and exercise therapy modules may be integrated into the system in order to alleviate a number of frequent MS symptoms. Rehabilitation is important in neurological diseases such as multiple sclerosis and can reduce a number of disease symptoms. By integrating exercise therapy modules into the present system, patients have training modules readily available, have control and visibility to their personal training plan and are therefore more motivated to perform their rehabilitation exercises. In a first step, a HCP assesses patient in person and prescribes therapy to patients account. In a second step, the HCP shows patient how to use the system and provides patient with a video monitoring and playback system such as the Microsoft Kinect™ system. In a third step, the patient can log in and perform his prescribed therapy while receiving immediate feedback from the system. In a fourth step, the HCP can later access their patient's progress online and make remote modifications as needed.

Other specific examples of rehabilitation and exercise therapy modules may include an application software program for finger muscle tone or joint stimulation by electrical stimulation or physiotherapy. A glove with built-in sensors can also be used for training muscle tone.

The patient therapy software and/or hardware components may also include a physiological monitoring module which is configured to measure the physiological state of a patient. The physiological module may comprise diagnostic interactive physiological tests such as vision tests, walking tests, mobility tests and muscular force test. The physiological monitoring module may also comprise a quality of life monitoring module which measures the patient's well-being against a scale.

In an embodiment, the physiological module may also comprise a physiological parameter analysing module, configured to receive a measured health parameter from a biosensor, said physiological parameter analysing module being configured to read and interpret physiological data selected from body temperature, blood pressure, pulse rate, galvanic skin response, surface electromyography, electroencephalography measurements, oculography measurements, electrocardiography measurements, breathing sensor measurements, blood sugar sensor measurements, urine markers and blood markers. The biosensor may be a wearable biosensor, preferably a bracelet or a patch biosensor which can be worn by the patient and is configured to continuously extract physiological data and transmit said data to the server system.

In particular for rheumatoid arthritis, a pressure sensor tool can be used. A stress ball with sensors can be used for measuring the grip strength of the patient. A joint functional measurement program can be used for measuring the swelling of joints by recording data of the presence of tender or swollen joints. The measurement program may comprise a visualisation of the joints in the body and request that the patient enters information whether the joint is tender and/or swollen.

Diagnostic therapy modules that enable patients to test vision at home transmit the captured data to the remote server are useful for diseases which include a degenerative eye disease. A HCP can review the vision results from a remote location in respect to the patient. Remote vision tests enable a quick and efficiently vision monitoring. Another benefit is that it is easy for the patient to perform vision tests at home and by more frequent measurements detect early changes in vision that may signal disease onset. The display of a handheld user interface device (UID) may be configured to display the test and the result achieved by the patient. By using vision application software and a shape discrimination test implemented on a user interface device (UID) for instance in the form of a handheld electronic device, the therapy module allows patients with degenerative eye disease to quickly and accurately test their own visual function at home.

Advantageously, measurement data from the physiological monitoring module may be used for physiological assessment of the patient as a first step in the other e-health therapy services in order to configure therapy treatment programs.

A dietary module may also be provided as a therapy module and can be beneficial for most diseases. In particular, for Crohn's disease and ulcerative colitis a specific diet is useful to reduce the disease symptoms. The therapy module can provide instantaneous feedback information to the patient upon entry of name/type of the foodstuff, whereby the dietary module will provide information if the foodstuff is bad, good, or questionable for their condition. The dietary module may also track calories and exercise.

The dietary module may also collect and store information about what the patient has eaten and gastrointestinal symptoms. The foodstuff, the time of intake and the gastrointestinal symptoms are inputted by the patient, whereby the dietary module is configured to correlate gastrointestinal symptoms to a certain type of foodstuff. The specific foodstuff that triggers the patient's gastrointestinal symptoms can be identified as “trigger foods” and stored in the user interface device. The dietary module can be activated to prompt the patient to adhere to the recommendations in particularly during disease flares.

Further examples of electronically assisted patient health therapy (e-health therapy) services comprise patient therapy software and/or hardware components including an information module configured to receive information requests from a user and to send responses on the user interface device (UID).

In an embodiment, the information module is configured to supply a user (in particular the patient) with drug information. The information can comprise dosages, instructions for administrations and potential side-effects. The medical device may also comprise a reader configured to recognize the drug located inside from information on the drug container and a processing unit configured to send identification information about the drug to the remote server. Additionally or alternatively, the user interface device or the health care professionals interface device can be configured to receive manually entered drug information and respond to the user interface device with drug related information. In an embodiment, the information module may comprise at least one downloadable instruction file and one video comprising instructions about use and administration of medication. However, if the patient is using several drugs, the information module may comprise information relating to all the drugs.

The information module may also comprise point of interest data such as location and contact information of physicians, physiotherapists, support groups, and specific facilities which are useful to the patients. In an embodiment, the user interface device is configured receive an information request and to send a measured GPS location point to the information database in order to retrieve a point of interest.

Examples of point of interest data may include data for finding a physician, such as a general practitioner or a specialist in the particular disease of the patient, a local support group or a pharmacy. The therapy services may also include information about devices and household appliances which are particularly adapted to the patient's disease. In particular for patients with Crohn's disease or ulcerative colitis, the system may also be configured to show the patient the closest toilet via GPS.

In an embodiment, the adherence to the therapy modules may be monitored such that the patient care software components are configured to receive patient therapy data from the patient therapy module and the reports component is further configured display adherence to the compliance measurement to the patient therapy modules.

The patient therapy software and/or hardware components may further comprise a central therapy software control module, configured to extract result information from individual modules and identify need of other training modules. As the efficacy of the therapy modules may be interrelated, the central therapy software control module may for instance identify the need of a second therapy module based on the results in a first module, or recommend a certain therapy module based on measurements from the physiological monitoring module.

In an embodiment, the patient therapy software and/or hardware component may also be configured to generate recommendations to the patient and/or health care professionals, based on data from the patient therapy software, whereby the recommendations can be sent by the notification services software component 30b to the patient and/or a health care professional.

Patient care system may also be configured such that the patient information management program is configured to analyse entered data from patient outcome reports, training modules and physiological measuring module and send triggers for further follow-up to a health care professional.

Optionally, the patient information management program may also comprise follow-up programs which can be selectively activated by a HCP, said follow-up programs comprising measurement of physiological parameters and pain ratings. Follow-up programs are especially useful at specific times, such as before or after a patient has received a specific drug (such as chemotherapy) or during a relapse of the disease.

Patient therapy services may further include alarms 40c, for instance alarms for administration of medication and alarms for visits to the treating clinic, the HCP, or the field nurse. The online capture of results of tests may be fed into the reports 32f component and the performance of training may also be recorded and available to the reports component or as information available to the HCP and filed nurse. Alarms may be sent by the notification services component 30b. Tests may be done by the patient at home using the patient's UID 18 and test apparatuses such as motion sensors connected to the UID 18 by a wired or wireless connection. The tests may be vision tests, walk tests, cognition tests and/or mobility tests. Patient physiological monitoring may also include physiological measurements of the patient's measured physiological data, such as body temperature, blood pressure, pulse rate, height (in the case of a growth hormone treatment) of the patient. Certain physiological measurements may be captured automatically by sensors and transmitted to the server system via the patient's UID 18 and/or medical device 1 an/or by the sensing or training device if it is equipped with communications means for data transfer over the internet 7 or the WTT network 3. Certain physiological data may be manually entered by the patient 15a or the HCP 15b or the field nurse 15c.

In an exemplary embodiment, the software components illustrated in FIGS. 4 and 5 and presented above may comprise the following features:

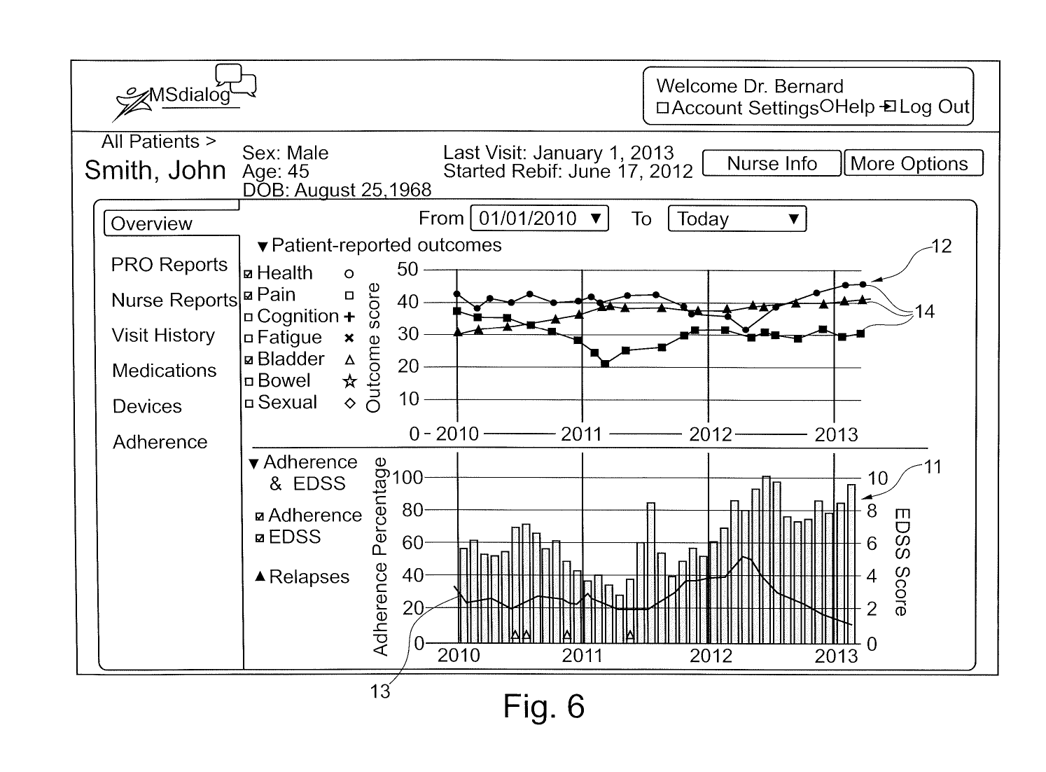

Therapy 36c—this software component provides functionality related to drug therapy which uses medication to treat the disease, in a particular embodiment multiple sclerosis disease. Therapy is associated with medication as medication is governed by a Therapy.
Medication 36b—this software component provide functionality related to medication prescribed or taken by the Patient. Medication component is associated with Therapy as Therapy governs the Medication given to patients. The medication 36b component may provide the patient with drug related information regarding medicines at the time they should be taken. The medication 36b component may also provide information about medication orders and their planned delivery date as well as reimbursement support from a third party, such as a health insurance. The medication component 36b may comprise all these features in a system which is also configured to support the online order and home delivery of drugs.
Device 36a—this software component provides functionality related to the medical device 1, in a specific embodiment the medical device is Rebismart™ used to inject Rebif™ medicine to treat multiple sclerosis disease. Device component 36a stores medical device usage data, for instance injection data, and is also associated with patient data 32b as every patient will have at least one device associated with him/her.
Injection 36d—this software component provides functionality to store the date and time of drug administration, for instance injections, and their related information (e.g. injected dose). In a specific embodiment, Injection is a method to give medicine to multiple Sclerosis patients. Injection data will be passed to Device component 36a.
Patient Data 32c—this software component provides maintenance of patient data which is gathered using Device component 36a and a web application.
Clinic 32a—this software component manages the association of a treating clinic to a patient 15a, in a specific embodiment a Multiple Sclerosis patient.
Consent 32e—this software component manages the consent of the patient 15a for being treated by a primary physician or other HCPs 15b whoever might be involved in treating the patient in the same clinic where patient is undergoing the treatment.
Patient Outcome 32d—this software component will provide the outcome of questionnaire presented to patient and will use Report component 32f for generating the health reports for a patient 15a, in a specific embodiment for a patient suffering from Multiple Sclerosis disease.
Patient Visit 32b—This software component manages the visit information of the patient entered by the HCP 15c and field nurse 15d. Visits help to track the EDSS Score, relapses and the hospitalization period of the patient since last visit.
Reports 32f—this software component generates patient reports from the data received through medical device usage received from the medical device that provides adherence data, questionnaires filled by the patient that may provide patient reported outcomes, adherence settings including information on the treatment regimen, and visit data included by the HCP and field nurse. Reports advantageously include composite diagrams that present in a single view multiple reports along a common time scale, including patient reported outcomes (PRO) and adherence information along a common time scale, as will be discussed in more detail further on and illustrated in FIG. 6. The afore mentioned composite diagram enables the HCP to more easily and quickly assess the effects of non-adherence of a patient to the prescribed treatment regime and if needed to propose corrective measures such as reminding the patient on importance of adherence, enhanced monitoring of adherence, increased frequency of visits, a modification in the treatment regime, and additional treatment or therapeutic measures. In an embodiment, the reports 32f may display historical records to understand disease development or history. The historical records may be accessible for both the HCP and the patient.
Calendar 32g—this software component will manage calendars for HCPs and patients for providing details on events as well as scheduled visits. Calendar software component 32g may also issue reminders of an event to patient or HCP.
Questionnaire 38d—this software component manages all the questionnaires presented to the patient and their responses. This feature may provide outcomes of surveys provided by the patient information management module 32 to the patients. The questionnaires include clinically validated questionnaires directed to the patient, the responses of whom are used by the Reports component 32f to generate patient reported outcome (PRO) reports. The questionnaire may contain questions permitting evaluating the level of general health, pain, temperature, cognition, fatigue, bladder condition, bowel condition, sexual satisfaction or other health criteria of the patient. The questionnaire may also contain questions related to experienced side-effects and the use of additional drugs.
User Management 38a—this software component involves managing user access rights of the various users (HCP, field nurse, patient, system administrator, key account manager and payer).
Audit Trails 38b—this software component provides audit trail management for all transactions executed in the patient care system by any user.
Configuration 38c—this software component is used for managing system configuration and TimeZone configuration e.g. country specific configuration.
E-Mail and SMS 30b—these software components send e-mail notifications, respectively SMS notifications to users of the patient care system.

In particular for Crohn's disease and ulcerative colitis, for the input of patient reported outcomes the patient can input their stool cycle, blood in their stool, diarrhoea, nocturnal diarrhoea, abdominal pain, bowel obstruction, weight loss, fever, night sweats, hematochezia, fever, weight loss, malaise, nausea, and arthralgias.

The E-Mail and SMS 30b for Crohn's disease and ulcerative colitis may be configured to send reminders for the time approaching a colonoscopy. The reminders may include a first reminder a few days before the colonoscopy to start eating a low-fiber food and indicate foods to avoid such as whole grains, nuts, seeds, dried fruit, or raw fruits or vegetables; A second reminder the day before and the day of the colonoscopy reminding the patient to not eat solid foods, but to consume only clear liquids; A third reminder in the afternoon or evening before the colonoscopy, reminding the patient to drink a liquid that will trigger bowel-clearing diarrhoea; A fourth reminder two hours before the procedure to not drink anything. Additionally, other reminders for preparation for colonoscopy may be added by a health care practitioner.

Certain of the software components described herein may be in the form of client—server applications whereby a client side application is installed on the user interface device (UID) to allow the UID to connect to the application on the server system and run certain features locally. Certain software components described herein may be in the form of browser applications that are accessed and run through a web browser on the users UID. Certain software components may also be installed and run independently on various devices of the system including medical devices, medical device connection stations, and user interface devices, depending on the type and function of the software application. The use of the term “software component” herein may mean a portion of a program, a program, a plurality of portions of programs or a plurality of programs that work together to achieve the desired function.

The integration of the aforementioned software components in the server system 6 communicating with client applications installed on both mobile and wired user interface devices 18 described herein provides a flexible global patient care system optimising the quality of information available to patients, HCPs, field nurses and other members of patient support organisations as well as other actors such as drug manufacturers to improve overall patient care and health, while reducing the healthcare costs. Moreover, over time, data accumulated in the database allows the HCPs and drug providers to gain a better understanding of the disease and the effects of treatment in order to improve the treatment regime and therapy. The patient care system according to the invention allows remote patient treatment monitoring for health professionals, provides information for decision making to HCP's, enables ease of administering medications for patients, provides easy health communication between HCPs and patients globally, and provides a safe, effective and economical patient care.

In an embodiment of the invention, the medical device 1 is an injection device for injecting liquid medicine, in particular Rebif® (interferon beta-1a) or Saizen® (recombinant growth hormone). The medical device 1 may for example be of the type, or include certain features of, the devices disclosed in WO 2005/077441, WO 2006/085175, WO 2006/085204 or WO 2007/088444 (which are incorporated herein by reference) and marketed under the registered trademarks RebiSmart and Easypod.

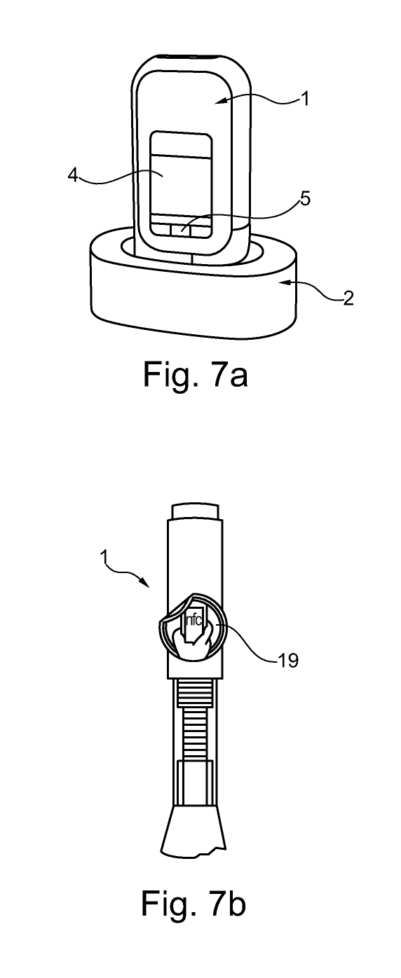

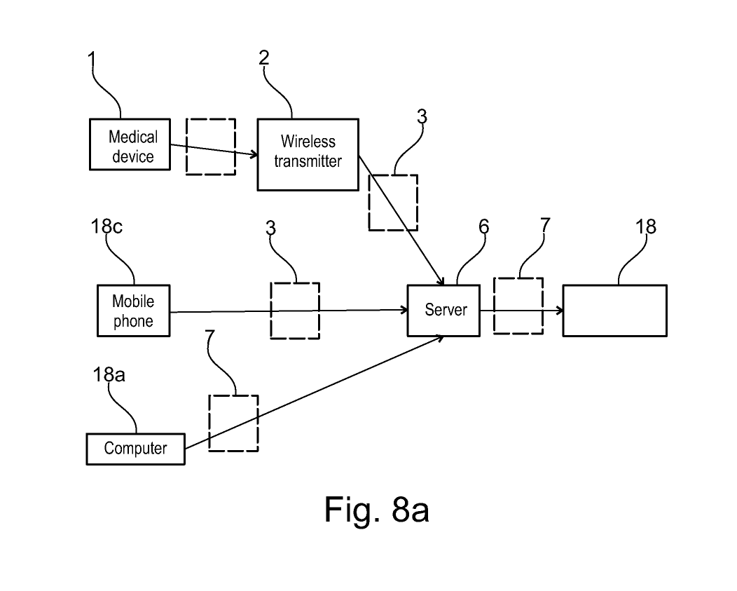

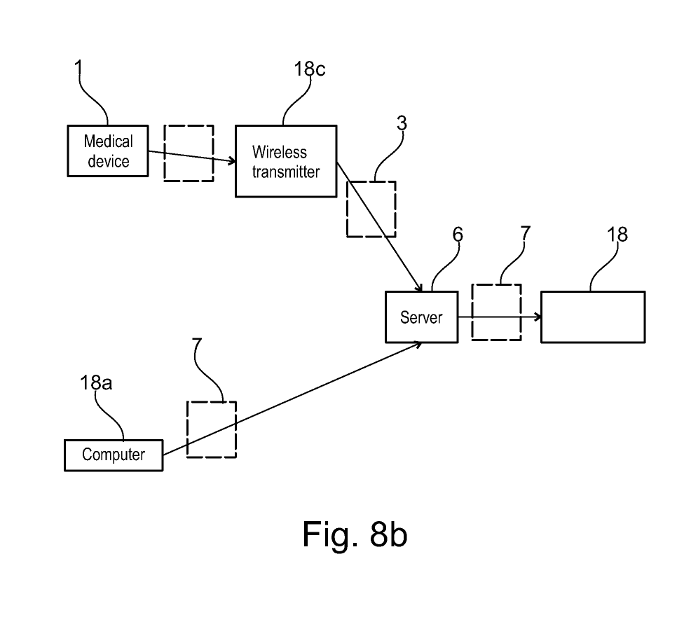

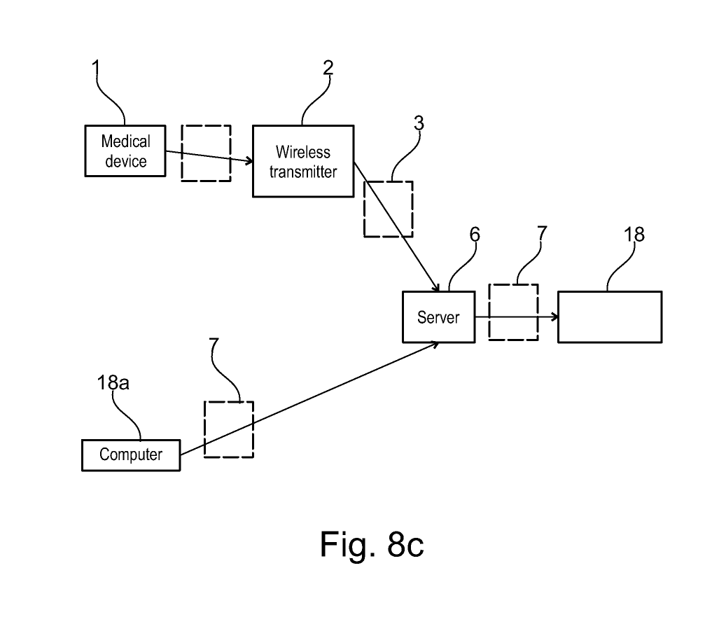

Referring more particularly to FIGS. 7a, 7b, 8a to 8c an example of the configuration of a patient care system according to an embodiment of the invention, especially for monitoring adherence to a treatment regime, for instance a treatment regime for patients suffering from Multiple Sclerosis or growth hormone deficiency, will now be described.

In the embodiment, the medical device 1 preferably includes an internal memory and processor which is programmed to store first data relating to the use of the medical device by the patient in the memory.

The first data includes medical device usage data, comprising the amount of medication administered and the date and/or time of administration. In a variant where the medical device is an injection device, first data may include the number of injections made, the times of the injections, and the doses injected. The internal memory and processor further stores an identification code that uniquely identifies the medical device 1.

In variants where the medical device does not comprise and internal memory and microprocessor to store the medical device usage data, the patient or person administering the medication may manually enter the first data in a user interface device 18 provided with a client software program generating a form for entry of the data and storage of the first data in a memory of the user interface device.

In an embodiment, the system also comprises a medical device connection station 2. As shown in FIG. 7a, the medical device connection station 2 may comprise a docking interface on which the medical device 1 may be positioned. The medical device connection station may include wireless telecommunications transfer electronics and a SIM (Subscriber Identity Module) card for connection to a mobile phone network 3 such as the GSM (Global System for Mobile communication) or the UMTS (Universal Mobile Telecommunications System). When placed on the medical device connection station 2, the medical device 1 communicates with the medical device connection station 2 by infrared, radiofrequency or electrical connection. Via a graphical user interface 4 and buttons 5 on the medical device 1, the patient may transmit the first data and the identification code from the medical device 1 to the medical device connection station 2. The first data and the identification code are then transmitted via the mobile phone network 3 to the distant server system 6.

The server system 6 acquires, stores and processes the first data. The data transmission between the medical device connection station 2 and the server system 6 is encrypted. The data is stored confidentially in the server system 6 and processed so as to be easily and securely accessible to the patient's care giver via a communication network such as the Internet 7. Thus, using a user interface device 18, typically a computer or a digital tablet, and via an encrypted communication, the physician may access the data stored in the server system 6 for his/her patient.

In addition to the aforementioned first data, the server system 6 acquires from the patient second data relating to a physiological state of the patient. These second data are sent to the server system 6 by a smart mobile phone 18c via the mobile phone network 3, by a computer 18a via the Internet network 7 and/or by any other terminal of the patient. The second data may include patient-reported outcomes (PROs), which may include responses to a clinically validated questionnaire previously sent by the server system 6 to the smart mobile phone 18c and/or the computer 18a upon request by the physician. The patient regularly, when prompted by his UID 18, or at will, answers the questionnaire and the answers are sent to the server system 6, which stores and processes them. The questionnaire may contain questions permitting evaluating the level of general health, pain, cognition, fatigue, bladder condition, bowel condition, sexual satisfaction or other health criteria of the patient. The second data may further include health test results. The tests may be done by the patient at home using the computer 18a and test apparatuses such as motion sensors connected to the computer 18a by e.g. a radiofrequency connection (e.g. Bluetooth® or Wi-Fi). The tests may be vision tests, walk tests, cognition tests and/or mobility tests. The second data may also include measured physiological data, such as the body temperature, the blood pressure, the height (in the case of a growth hormone treatment) of the patient, blood parameters, urinalysis or ECG data. Like the first data, the second data is transmitted in an encrypted manner and confidentially stored in the server system 6.

When the physician connects his/her terminal 8 to the server system 6, he/she may access the first and second data stored therein for the patient. According to the present invention, and as shown in FIG. 6, two graphical representations 11, 12 respectively obtained from the first and second data stored in the server system 6 are used to generate a composite report, whereby first and second data are simultaneously displayed on the screen of the terminal 8 so that they can be viewed together. By the term “simultaneously” it is meant that there exists at least a period of time during which the two graphical representations 11, 12 are displayed together. The two graphical representations 11, 12 may be side-by-side, superimposed or one above the other as illustrated.

The first graphical representation 11 represents the adherence to the medical treatment, in particular the extent to which the patient follows the medical instructions such as dose and injection frequency, as a function of time. Thus, an adherence of 100% indicates that the patient has strictly followed the medical prescription, while an adherence lower than 100% indicates that the patient did not inject all prescribed doses. The first graphical representation 11 may for instance be in the form of a bar graph, as illustrated, and may be combined with a curve 13 representing a condition of the patient, for instance in the case of a neurodegenerative disease such as Multiple Sclerosis, the EDSS (Expanded Disability Status Scale) score of the patient which is provided to the server system 6 by the physician. The second graphical representation 12 may advantageously include one or several curves 14 respectively representing the aforementioned patient-reported outcomes as a function of time. The physician may select which patient-reported outcome(s) to display. Although not shown, the second graphical representation 12 may also include curves or other graphical data representing the health test results and the measured physiological data.

Since the graphical representations 11, 12 are simultaneously displayed on the physician's terminal 18, the physician may easily analyse the data, and may observe the influence of the adherence on the health of the patient. The physician may then evaluate the efficacy of the medical treatment, adjust the prescription and/or incite the patient to more strictly follow the prescription, and/or propose accompanying therapeutic measures.

The adherence may be calculated by the software components in the server system 6 based on the first data provided by the medical device 1 and the medical device connection station 2. The first and second graphical representations 11, 12 are also produced by software components in the server system 6. However, in a variant, a client software application installed on the physician's computer terminal 18 could calculate the adherence and produce the graphical representations 11, 12 based on raw data provided by the server system 6. In another variant, the adherence could be calculated by a software component in the server system 6 and the graphical representations 11, 12 could be produced by a client software application installed on the computer terminal 18. In still another variant, the adherence could be calculated by a software component in the medical device 1.

The first data may be sent by the server system 6 to the mobile phone 18c, the computer 18a and/or any other terminal of the patient upon request by the physician or automatically, thus enabling the patient to monitor the adherence.

In a variant of the invention, shown in FIGS. 8b and 7b, the medical device 1 is a fully mechanical injection device such as the Rebidose®, but bears a smart label 19. The smart label 19 typically includes an NFC (Near Field Communication) chip and a QR (Quick Response) code. The identification code of the medical device is stored in the NFC chip and, also, represented by the QR code. If the smart mobile phone 18c is equipped with NFC means, tapping the smart mobile phone 18c to the medical device 1 triggers transmission of the identification code into the smart mobile phone 18c. Otherwise, the identification code may be accessed by the smart mobile phone 18c by reading the QR code. The patient then performs the injection with the medical device 1 and confirms the injection to the smart mobile phone 18c, which can in turn transmit the injection data (the aforementioned “first data”) and the identification code to the server system 6. Thus, in this variant, the mobile phone 18c and the transmitter are one and a same device. The data processing which, in the embodiment of FIG. 1, was performed by the medical device, is here performed by the smart mobile phone 18c.

A further variant of the invention is shown in FIG. 3. Here, the medical device 1 is integrated with the first data input device and the second data input device. The medical device 1 acquires and stores the first and second data. The medical device docks to a wireless medical device connection station 2 and communicates the data to the medical device connection station via infrared. The medical device connection station 2 transmits the data to the server system 6 via a mobile network 3. A computer 18a belonging to the patient acquires second data, relating to a physiological state of the patient, and transmits it to the server system 6 via the internet 7. The server system 6 stores and processes the first and second data and transmits it to the care giver's computer 18 via the internet 7.

The present invention may also apply to other medical devices than those shown in FIGS. 7a, 7b, for example to electronic pill dispensers or pill dispensers having smart blisters. The patient may in fact need to use different medical devices depending on the medical treatments he/she follows. Since each medical device has a unique identification code, the server system 6 knows which medical device the acquired first data is concerned with.

The server system 6 is not necessarily located at one place but may be comprised of several distant servers communicating with each other through telecommunication networks. For example, the server system 6 may be comprised of a first server which stores and processes the data and a web server which makes the data available to the physicians through the Internet.

Finally, the skilled person will recognise that the medical device connection station 2 is not necessarily be a separate device but could be integrated in the medical device 1.

The computerized patient care system according to the invention may be referred to as an eHealth medical platform. Advantageously, the platform is secure and modular. The platform provides a set of applications to share information among care givers and patients in order to monitor and act upon a patient's condition, adherence to a dosage regimen and progress of a disease.

The invention enables a care giver or a patient to monitor the adherence to a medical treatment regimen and to monitor the efficacy of a medical treatment regimen.

The patient care system according to the invention has numerous benefits, including:
the ability to inform the patient if their adherence is low and if they are not uploading often enough provision of coaching aides, or to provide advance warning such as telling the patient to take cognition training, rest or take a cold shower if their patient reported outcomes (PROs) show that they might relapse, and therefore help to prevent relapse by improving adherence and providing coaching
patient support groups and field nurses will have access to the patients history of adherence and PROs they will be able to provide more tailored coaching to the patient
the physician is better prepared for the patent's visit
if adherence is low, the physician can link low adherence to side effects, which can then be better managed
adherence in many diseases, in particular multiple sclerosis, has never been correlated to PROs so the system will help us to learn more about the disease, for instance the conditions leading to relapse and how it can be avoided.

1.-61. (canceled)

62. A patient care system comprising a medical device (1) for administering a medical treatment to a patient (15a) and a server system (6) configured to receive and transmit data via a communications network (16) to and from users including patients (15a) and health care professionals (15b), the server system further configured to process and store data related to patient care, wherein the server system comprises a database (6c) configured to store data related to patient care, an application server (6b) including patient care software components for disease management (36) and patient information management program (32), a communication server (6a) including a web server software application for data transfer through the internet, the patient information management program comprising a reports component configured to generate reports, in the form of tables, charts, lists, diagrams or graphical representations based on information selected from drug administration history, adherence data, patient outcome reports, patient health reports, patient physiological data reports, medical device settings, treatment regimen data, and any combination of aforesaid information, the patient care software components configured to receive medical device usage data comprising data on the usage of said medical device transferred through the communications network, and further configured to process said medical device usage data in conjunction with patient data (32c) to generate a report (32f) or a plurality of reports related to the treatment of the patient, the reports being accessible remotely via the communications network by registered users of the patient care system as a function of respective roles and privileges of the registered user stored in the server system, the patient care system further comprising a patient therapy software and/or hardware component having at least one interactive module selected from the group of physiological monitoring modules, training modules, information modules and psychological monitoring modules.

63. The patient care system according to claim 62, wherein, the reports component is configured to form composite reports for simultaneous display on a user interface device (UID) display, including composite adherence and patient outcome reports to facilitate evaluation of the effects of non-adherence to the treatment regimen or the efficacy of the medical treatment.

64. The patient care system according to claim 62, wherein the patient therapy software is a web-based program which resides on the application server.

65. The patient care system according to claim 62, wherein the patient therapy software resides on the patient's user interface device or on the medical device.

66. The patient care system according to claim 62, wherein the reports comprise information from the patient therapy software.

67. The patient care system according to claim 62, wherein the patient care software components are configured to receive patient therapy data from the patient therapy module and reports component is further configured display adherence to the compliance measurement to the patient therapy modules.

68. The patient care system according to claim 62, further comprising a central therapy software control module, configured to extract result information from individual modules and identify need of other training modules.

69. The patient care system according to claim 62, wherein the server system further comprises a notification services software component (30b) configured to transmit email and/or SMS (Short Message Service) notifications to patients and optionally other users of the system.

70. The patient care system according to claim 69, wherein the patient therapy software and/or hardware component is configured to generate recommendations to the patient and/or health care professionals, based on data from the patient therapy software, said recommendations being sent by notification services software component to the patient and/or health care professionals.

71. The patient care system according to claim 62, wherein the patient therapy software and/or hardware components comprises a cognitive remediation training module, the cognitive remediation training module being accessible from the user interface device (UID), the cognitive remediation training module comprising a processor configured to run a patient adapted program with training exercises, a results analyser and a memory to store past results.

72. The patient care system according to claim 62, wherein the patient therapy software and/or hardware components comprises a cognitive depression management module, the cognitive depression management module being accessible from the user interface device (UID) and comprises a depression evaluation algorithm, a processor configured to run a patient adapted program with training exercises configured based on data from the depression evaluation algorithm, results analyser and a memory to store past results.

73. The patient care system according to claim 62, wherein the patient therapy software and/or hardware components comprises a cognitive sleep management module, the cognitive sleep management module being accessible from the user interface device (UID) and comprises training exercises, a results analyser and a memory to store past results.

74. The patient care system according to claim 71, wherein the processor is configured to iteratively adapt the cognitive training program during a training session, such that the program can change based on the information entered by the patient.

75. The patient care system according to claim 62, wherein the server system further comprises patient therapy software and/or hardware components including a physiological monitoring module, configured to measure the physiological state of a patient.

76. The patient care system according to claim 62, wherein the physiological monitoring module comprises at least one interactive physiological test selected from the group of vision tests, walking tests, mobility tests and muscular force test.

77. The patient care system according to claim 75, wherein the patient therapy software and/or hardware components includes a physiological parameter analysing module, configured to receive a measured health parameter from a biosensor, said physiological parameter analysing module being configured to read and interpret physiological data selected from body temperature, blood pressure, pulse rate, galvanic skin response, surface electromyography, electroencephalography measurements, oculography measurements, electrocardiography measurements, breathing sensor measurements, blood sugar sensor measurements, urine markers and blood markers.

78. The patient care system according to claim 77, wherein the biosensor is a wearable biosensor, preferably a bracelet or a patch biosensor which can be worn by the patient and is configured to continuously extract physiological data and transmit said data to the server system.

79. The patient care system according to claim 62, wherein the server system further comprises patient therapy software and/or hardware components comprises an activity tracking module configured to track an amount of exercise performed.

80. A method of monitoring treatment and providing care to a patient suffering from a chronic disease or condition, comprising:
a) providing a medical device for administering a treatment drug to the patient,
providing a computerized patient care system comprising a server system configured to receive and transmit data via a communications network to and from users including patients and health care professionals, the server system comprising a database configured to store data related to the patient, an application server including patient care software components for disease management and patient information management, a communication server for data transfer through the communications network, the patient care software components further comprising a patient therapy software and/or hardware component,
b) processing and storing data related to patient care on the server system,
c) transferring medical device usage data comprising data on the usage of said medical device through the communications network to the server system,
d) transferring usage data of the patient therapy software and/or hardware component through the communications network to the server system, and
e) processing said medical device usage data and patient therapy software and/or hardware component usage data in conjunction with patient data to generate a report or a plurality of reports related to the treatment of the patient, providing remote access to the reports via the communications network to registered users of the patient care system as a function of respective roles and privileges of the registered users stored in the server system.

81. Method for evaluating the efficacy of a treatment regimen for a chronic condition or disease comprising:
a) providing a medical device to deliver a medicament,
b) providing at least one therapy software on a user interface display,
c) transmitting usage data of the medical device to a computing system,
d) transmitting adherence to the therapy software module
e) calculating adherence to a treatment regimen based on a prescribed treatment regimen data and said usage data,
f) transmitting to the computing system a patient reported outcome,
g) generating in the computing system a report comprising a first graphical representation of the adherence to a treatment regimen and a second graphical representation of the patient reported outcome, said first and second graphical representations comprising a common time scale
h) rendering said report accessible to a health care professional for display on a screen of a user interface device, wherein said first and second graphical representations are simultaneously displayed.
